SEQUENCE LISTING (18 sequences)

SEQ ID NO: 1 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 3 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 4 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 5 - protein, 10 aa, Artificial Sequence
  Description of Artificial Sequence Synthetic peptide
GFTFSRYWMS

SEQ ID NO: 6 - protein, 17 aa, Artificial Sequence
  Description of Artificial Sequence Synthetic peptide
NIKQDGSEKYYVDSVKG

SEQ ID NO: 7 - protein, 12 aa, Artificial Sequence
  Description of Artificial Sequence Synthetic peptide
EGGWFGELAFDY

SEQ ID NO: 8 - protein, 12 aa, Artificial Sequence
  Description of Artificial Sequence Synthetic peptide
RASQRVSSSYLA

SEQ ID NO: 9 - protein, 7 aa, Artificial Sequence
  Description of Artificial Sequence Synthetic peptide
DASSRAT

SEQ ID NO: 10 - protein, 9 aa, Artificial Sequence
  Description of Artificial Sequence Synthetic peptide
QQYGSLPWT

SEQ ID NO: 11 - protein, 139 aa, Artificial Sequence
  Description of Artificial Sequence Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 12 - protein, 167 aa, Artificial Sequence
  Description of Artificial Sequence Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 13 - protein, 10 aa, Artificial Sequence
  Description of Artificial Sequence Synthetic peptide
GFTFSSYGMH

SEQ ID NO: 14 - protein, 15 aa, Artificial Sequence
  Description of Artificial Sequence Synthetic peptide
VIWYDGSNKYYADSV

SEQ ID NO: 15 - protein, 16 aa, Artificial Sequence
  Description of Artificial Sequence Synthetic peptide
DPRGATLYYYYYGMDV

SEQ ID NO: 16 - protein, 11 aa, Artificial Sequence
  Description of Artificial Sequence Synthetic peptide
RASQSINSYLD

SEQ ID NO: 17 - protein, 7 aa, Artificial Sequence
  Description of Artificial Sequence Synthetic peptide
AASSLQS

SEQ ID NO: 18 - protein, 9 aa, Artificial Sequence
  Description of Artificial Sequence Synthetic peptide
QQYYSTPFT

(19) United States
(12) Patent Application Publication
(10) Pub. No.: US 2022/0332828 A1
(43) Pub. Date: Oct. 20, 2022

(54) ANTI-PD-L1 ANTIBODY TREATMENT OF BLADDER CANCER

(71) Applicant: MEDIMMUNE, LLC, GAITHERSBURG, MD (US)

(72) Inventors: JOHN KURLAND, GAITHERSBURG, MD (US); JOHN ANDREW BLAKE-HASKINS, GAITHERSBURG, MD (US); MAGDALENA ZAJAC, CAMBRIDGE (GB); MARLON REBELATTO, GAITHERSBURG, MD (US); ASHOK GUPTA, GAITHERSBURG, MD (US); TONY HO, GAITHERSBURG, MD (US); JILL WALKER, CAMBRIDGE (GB); XIAOPING JIN, GAITHERSBURG, MD (US); SHANNON MORRIS, GAITHERSBURG, MD (US); ROBERT IANNONE, GAITHERSBURG, MD (US); LI SHI, GAITHERSBURG, MD (US); MOHAMMED DAR, GAITHERSBURG, MD (US); YONG BEN, GAITHERSBURG, MD (US)

(21) Appl. No.: 17/720,903

(22) Filed: Apr. 14, 2022

Related U.S. Application Data
(63) Continuation of application No. 16/486,222, filed on Aug. 15, 2019, now abandoned.
(60) Provisional application No. 62/459,700, filed on Feb. 16, 2017.

(51) Int. Cl. C07K 16/28; A61P 35/00; A61K 9/00; G01N 33/542

(52) U.S. Cl. CPC C07K 16/2827; A61P 35/00; A61K 9/0019; C07K 16/2818; G01N 33/542

(57) ABSTRACT

Provided are methods of treating bladder cancer (e.g., urothelial carcinoma, UC) in a subject having bladder cancer, e.g., UC, with an effective dose regimen of an anti-PD-L1 antibody, e.g., durvalumab, or an antigen binding fragment thereof. Also provided are methods in which an anti-PD-L1 antibody is used in combination with another immunotherapeutic agent, e.g., tremelimumab to treat a bladder cancer, e.g., UC, in a subject having bladder cancer. In some cases, the subject undergoing treatment is identified as having a bladder cancer or tumor that is PD-L1-low/neg, or PD-L1-high. Methods are also provided in which anti-PD-L1 antibody treatment of bladder cancer is used following a standard of care or first-line therapy in subjects who have progressed following such therapies or who have relapsed after a prior treatment regimen.

### FIELD OF THE INVENTION

The present invention generally relates to methods of treating bladder cancer in a subject having cancer of the bladder with an effective dose regimen of an anti-PD-L1 antibody, alone or in combination with one or more additional anti-cancer agents or therapies.

### BACKGROUND OF THE INVENTION

Bladder cancer is the ninth most common cancer diagnosis worldwide, with an estimated 429,800 new cases each year and 165,100 cancer-related deaths reported globally in 2012. In the United States, based on medical statistics, 76,690 new cases will be diagnosed and 16,390 deaths are likely to occur in 2016. The most common type of cancer of the bladder, ureter, urethra, and urachus is urothelial carcinoma (UC), also known as transitional cell carcinoma (TCC), which accounts for approximately 90% of primary malignancies of the urinary tract. More than 90% of urothelial tumors originate in the urinary bladder, while 8% originate in the renal pelvis, and 2% originate in the ureter and urethra.

Bladder cancer is generally divided into muscle-invasive and non-muscle-invasive disease, based on invasion of the muscularis propria. At the initial diagnosis of bladder cancer, 70% of cases are diagnosed as non-muscle-invasive bladder cancer (NMIBC) and approximately 30% as muscle-invasive-bladder cancer (MIBC). Up to 40% of patients with NMIBC progress to MIBC within 5 years after diagnosis. Of patients presenting with MIBC at diagnosis, approximately 15% will have locally advanced disease and approximately 8% will have metastatic disease. The prognosis for patients with locally advanced or metastatic UC is poor, with a relative 5 year survival rate of approximately 15%.

Locally advanced or metastatic bladder cancer, particularly UC, is a life-threatening disease, with a large unmet need for new and effective treatment options, especially in patients who have cancer that has progressed during or after a first line of cancer treatment, such as a standard-of-care regimen.

Citation or identification of any reference in any section of this application shall not be construed as an admission that such reference is available as prior art to the present invention.

### SUMMARY OF THE INVENTION

As described below, the present invention features immunotherapeutic methods of treating cancer of the bladder, and/or bladder-associated structures, accessory organs and tissues, including the ureter, urethra, and urachus, by administering an anti-PD-L1 antibody in an effective dose regimen to a subject having such a cancer. In an embodiment, bladder cancer is urothelial carcinoma (UC), also known as urothelial cell carcinoma (UCC), or transitional cell carcinoma (TCC). In some embodiments, the subject suffers from locally advanced UC or metastatic UC. In a particular embodiment, the anti-PD-L1 antibody is durvalumab (also called MED14736 herein), which is a human monoclonal antibody (mAb) that hinds to PD-L1 and blocks its interaction with PD-1 and cluster of differentiation (CD) 80 (CD80) (B7.1). In another embodiment, the anti-PD-L1 antibody may be administered in conjunction with another therapeutic or chemotherapeutic agent, such as an anti-CTLA4 antibody (e.g., tremelimumab) or an antigen binding fragment thereof, platinum, and the like, or in conjunction with another anti-cancer therapy, such as radiation, surgery, chemotherapy, and the like. It will be understood that when reference is made herein to an anti-PD-L1 antibody, (e.g., durvalumab), or to an anti-CTLA4 antibody, (e.g., tremelimumab), such an antibody also embraces an antigen-binding fragment of the antibody which retains and exhibits specific binding to its target antigen. Use of an anti-PD-L1 antibody in effective amounts in the present methods provides both first-line (1L) treatment for a subject with bladder cancer, such as UC, and an effective second-line (2L) treatment for a subject with bladder cancer who has progressed following first-line therapy with standard-of-care (SoC) cancer treatment, e.g., containing a platinum drug, or who has relapsed following another therapy regimen.

In an aspect, the methods described herein are suitable for treating a subject having a bladder cancer or tumor, such as UC, wherein cancer or tumor cells or tissue derived from the bladder cancer are identified as expressing low, low-to-negative (low/neg), or high levels of PD-L1, for example, a PD-L1-low/neg, or PD-L1-high cancer or tumor, respectively. Methods for characterizing a subject's bladder cancer for levels of PD-L1 expression may therefore efficiently identify subjects as having tumors expressing low, negative-to-low (low/neg), or high levels of PD-L1, thereby directing the subject for appropriate and effective treatment with an anti-PD-L1 antibody, such as durvalumab, or with durvalumab in combination with another therapeutic agent, for example, an anti-CTLA4 antibody, such as tremelimumbab, and/or another anti-cancer agent or therapeutic. By way of example, reduced, low, as well as low/neg, PD-L1expression refers to about or equal to 5% to about 50%, or about or equal to 5% to about 25%, or about 25%, or 25% of the cancer or tumor cells or tissue derived from a subject's bladder cancer or tumor detected as expressing PD-L1. In other examples, reduced or low/neg levels of PD-L1expression refer to fewer than about 5% to 50%, or fewer than about 5% to 25%, or fewer than about 25%, or fewer than 25% of the cells or tissue derived from a subject's bladder cancer or tumor detected as expressing PD-L1 or as exhibiting staining with a reagent that detects PD-L1, such as an anti-PD-L1 antibody, etc. In a particular embodiment, reduced or low/neg levels of PD-L1 expression refer to fewer than 25% of the cells or tissue derived from a subject's bladder cancer, tumor, or tissue detected as expressing PD-L1. In a particular embodiment, high levels of PD-L1 expression refer to greater than 25% of the cells or tissue derived from a subject's bladder cancer, tumor, or tissue detected as expressing PD-L1.

In another aspect, a method of treatment is provided in which an anti-PD-L1 antibody is administered to subject having a bladder cancer in an effective amount to treat the bladder cancer. According to this aspect, the anti-PD-L1 antibody serves as a first-line cancer therapy, e.g., a monotherapy, for treating the bladder cancer. In an embodiment, the anti-PD-L1 antibody is durvalumab. In an embodiment, the subject's bladder cancer embraces bladder-associated structures and tissue comprising the ureter, urethra, urachus and/or the renal pelvis. In an embodiment, the subject's bladder cancer is urothelial carcinoma (UC), advanced UC, or metastatic UC. In an embodiment, the subject is identified as having bladder cancer, e.g., UC, with low/neg PD-L1 expression. In another embodiment, the subject is identified as having bladder cancer, e.g., UC, with high PD-L1 expression. In an embodiment, the anti-PD-L1antibody is administered in conjunction with another therapeutic or chemotherapeutic agent, such as an anti-CTLA4 antibody (e.g., tremelimumab), platinum, a platinum-containing cancer drug, and the like. In an embodiment, durvalumab is administered in an effective amount of 10 mg/kg to 30 mg/kg, or 10 mg/kg to 20 mg/kg, or 10 mg/kg, or 20 mg/kg every week (Q1W), every two weeks, (Q2W), every three weeks (Q3W), every four weeks (Q4W), every five weeks (Q5W), every six weeks (Q6W), every seven weeks (Q7W), every eight weeks (Q8W) up to every three months. In one embodiment, durvalumab is administered to a subject with bladder cancer such as UC in an effective amount of 10 mg/kg Q2W as a bladder cancer therapeutic. In another embodiment, durvalumab is administered to a subject with bladder cancer such as UC in an effective amount of 10 mg/kg Q4W as a bladder cancer therapeutic. In another embodiment, durvalumab is administered to a subject with bladder cancer such as UC in an effective amount of 20 mg/kg Q2W as a bladder cancer therapeutic. In another embodiment, durvalumab is administered to a subject with bladder cancer such as UC in an effective amount of 20 mg/kg Q4W as a bladder cancer therapeutic.

In another aspect, a method of treatment is provided in which an anti-PD-L1 antibody is administered to subject having a bladder cancer, e.g., UC, wherein the subject has progressed following a first-line (1L) therapy regimen, for example, platinum or cisplatin therapy, or a platinum drug containing combination therapy, or wherein the subject has relapsed within a given time period, for example, one year, after receiving neoadjuvant or adjuvant therapy, in which the anti-PD-L1 antibody is administered in an effective amount to treat the bladder cancer. As will be appreciated by the skilled practitioner in the art, adjuvant therapy refers, for example, to an anti-cancer or tumor therapy, such as chemotherapy or radiotherapy, following surgery for a cancer or tumor, to help decrease the risk of cancer or tumor recurrence. Neoadjuvant therapy refers to the administration of one or more therapeutic agents prior to a main or primary cancer treatment. For example, a neoadjuvant hormone therapy may be given prior to radiotherapy for treatment of a given cancer or tumor. According to this aspect, the anti-PD-L1 antibody can serve as a second-line cancer therapy for treating the bladder cancer following the first-line treatment, e.g., a platinum drug treatment. In an embodiment, the anti-PD-L1 antibody is durvalumab. In an embodiment, the subject's bladder cancer is UC, including advanced or metastatic UC. In an embodiment, the subject is identified as having bladder cancer, e.g., UC, with negligible to low/neg PD-L1 expression. In another embodiment, the subject is identified as having bladder cancer, e.g., UC, with high PD-L1 expression. In an embodiment, the anti-PD-L1antibody is administered in conjunction with another therapeutic or chemotherapeutic agent, such as an anti-CTLA4 antibody (e.g., tremelimumab), platinum, and the like. In an embodiment, durvalumab is administered in an effective amount of 10 mg/kg to 50 mg/kg, or 10 mg/kg to 30 mg/kg, or 10 mg/kg to 20 mg/kg, or 10 mg/kg, or 20 mg/kg every week, every two weeks, (Q2W), every three weeks (Q3W), every four weeks (Q4W), every five weeks (Q5W), every six weeks (Q6W), every seven weeks (Q7W), every eight weeks (Q8W) up to every six months, or longer. In one embodiment, durvalumab is administered to a subject with bladder cancer such as UC in an effective amount of 10 mg/kg Q2W as a bladder cancer therapeutic. In another embodiment, durvalumab is administered to a subject with bladder cancer such as UC in an effective amount of 10 mg/kg Q4W as a bladder cancer therapeutic. In another embodiment, durvalumab is administered to a subject with bladder cancer such as UC in an effective amount of 20 mg/kg Q2W as a bladder cancer therapeutic. In another embodiment, durvalumab is administered to a subject with bladder cancer such as UC in an effective amount of 20 mg/kg Q4W as a bladder cancer therapeutic.

In another aspect, a method is provided for treating a bladder cancer, e.g., UC, in a subject having bladder cancer, e.g., UC, and in need thereof, involving administering durvalumab to the subject after the subject has been identified as having a bladder cancer that is low to negative (PD-L1-low/neg), or high (PD-L1-high) for PD-L1 expression, in an amount effective to treat the bladder cancer. In an embodiment, the durvalumab is administered in conjunction with another therapeutic agent, for example, an immunotherapeutic agent such as an anti-CTLA4 antibody, for example, tremelimumab or an antigen binding fragment thereof. In an embodiment, durvalumab is administered as a second line therapy to a subject who has progressed following a first-line (1L) therapy regimen, for example, platinum or cisplatin therapy, or a platinum drug containing combination therapy, or who has relapsed within a given time period, for example, a year, after neoadjuvant or adjuvant therapy treatment. In an embodiment, durvalumab is administered in an amount of 10 mg/kg Q2W, 10 mg/kg Q4W, 20 mg/kg Q2W, or 20 mg/kg Q4W. In an embodiment, durvalumab is administered by intravenous administration.

In a particular aspect, a method of treatment is provided involving administering to a subject having a bladder cancer, e.g., UC, durvalumab in an amount of 10 mg/kg every 2 weeks (Q2W). In an embodiment, durvalumab is administered as an intravenous (IV) infusion. In an embodiment, the IV infusion occurs over 30 to 90 minutes. In a particular embodiment, the IV infusion occurs over 60 minutes. In an embodiment, the subject suffers from UC, locally advanced UC, or metastatic UC. In an embodiment, the subject has locally advanced or metastatic UC that has progressed during or after standard-of-care (SoC) treatment, e.g., after one standard platinum-based treatment regimen. In an embodiment, 10 mg/kg durvalumab is administered Q2W over 60 minutes by IV infusion to a subject whose bladder cancer, such as UC, has locally advanced or is metastatic, until a response, e.g., ORR, OS, PFR, or CR, is obtained in the subject, until disease progression, or until unacceptable toxicity is reached.

In another aspect, a method of treatment is provided involving administering to a subject having a bladder cancer, e.g., UC, an amount of about 10 mg/kg to 50 mg/kg of durvalumab or an antigen binding fragment thereof in combination with an amount of about 1 mg/kg to 10 mg/kg of tremelimumab or an antigen binding fragment thereof. In an embodiment, the subject is identified as having a bladder cancer, e.g., UC, which is low/neg, for expression of PD-L1. In an embodiment, the durvalumab is administered to the subject at a dose of 10 mg/kg. In an embodiment, the durvalumab is administered to the subject every 2 weeks (Q2W). In an embodiment, the durvalumab is administered to the subject at a dose of 10 mg/kg Q2W. In an embodiment, the durvalumab is administered to the subject at a dose of 1.5 g. In an embodiment, the durvalumab is administered to the subject every 4 weeks (Q4W). In an embodiment, the durvalumab is administered to the subject at a dose of 1.5 g Q4W. In an embodiment, the durvalumab is administered to the subject via intravenous infusion. In an embodiment, the durvalumab is administered to the subject via intravenous infusion over a 60 minute time period.

In certain aspects, tremelimumab or an antigen-binding fragment thereof is administered to a subject at a dose of about 1 mg/kg, or at a dose of about 3 mg/kg, or at a dose of about 10 mg/kg. In some embodiments, the subject is administered at least two doses of tremelimumab or an antigen-binding fragment thereof, wherein the dose is about 1 mg/kg, or about 3 mg/kg, or about 10 mg/kg. In some embodiments, the at least two doses are administered about four weeks apart or about twelve weeks apart. In other embodiments, the subject is administered at least three doses of tremelimumab or an antigen-binding fragment thereof, wherein the dose is about 1 mg/kg, or about 3 mg/kg, or about 10 mg/kg. In some embodiments, the at least three doses are administered about four weeks apart or about twelve weeks apart.

Further provided is a method of treating a bladder cancer in a subject with bladder cancer in which durvalumab or an antigen binding fragment thereof is administered to the subject in an amount of 50 to 2000 mg every 2-4 weeks (Q2W to Q4W), in combination with administering to the subject tremelimumab or an antigen binding fragment thereof in an amount of 50 to 150 mg every 2 to 4 weeks to treat the bladder cancer. In an embodiment, the durvalumab or an antigen binding fragment thereof is administered in an amount of 1500 mg every 4 weeks (Q4W) and the tremelimumab or an antigen binding fragment thereof is administered in an amount of 75 mg every 4 weeks (Q4W). In an embodiment, the durvalumab and the tremelimumab or antigen binding fragments thereof are administered for up to four doses per cycle. In certain embodiments, the durvalumab and the tremelimumab or antigen binding fragments thereof are administered at the same time or at different times. In an embodiment, the subject is identified as having a bladder cancer with a negligible to low expression of PD-L1 (PD-L1-low/neg). In an embodiment, the subject is identified as having a bladder cancer with a high level of expression of PD-L1 (PD-L1-high). In embodiments, the bladder cancer is urothethial carcinoma (UC) and/or cancer of bladder-associated structures and tissue comprising ureter, urethra, urachus and/or renal pelvis. In a particular embodiment, the bladder cancer is urothelial carcinoma, advanced UC, or metastatic UC.

In various embodiments of any of the above aspects or any aspect of the methods delineated herein, the bladder cancer encompasses cancer of any portion of the bladder, e.g., muscle or epithelium, as well as ductal structures such as the ureter, or the urethra, or the urachus. Bladder cancer subject to treatment by the methods described herein also include histologically or cytologically confirmed inoperable or metastatic transitional cell (including transitional cell and mixed transitional cell/non-transitional cell histologies) carcinoma of the urothelium (including the urinary bladder, ureter, urethra, and renal pelvis). In various embodiments of any of the above aspects or any aspect of the methods delineated herein, the anti-PD-L1 antibody is durvalumab (MEDI4736). In other embodiments, the anti-CTLA4 antibody which may be co-administered with durvalumab is tremelimumab. In various embodiments of any of the above aspects, the bladder cancer is muscle-invasive or non-muscle-invasive, based on invasion by cancer cells of the muscularis propria. In a particular embodiment of any of the above aspects, the bladder cancer is urothelial carcinoma (UC). In various embodiments of any of the above aspects, the treatment is administered every 2 weeks, every 3 weeks, every 4 weeks, every 5 weeks, every 6 weeks, every 7 weeks, or every 8 weeks. In various embodiments of any of the above aspects, durvalumab is administered in an amount of 10 mg/kg every 2 weeks or every 4 weeks; or in an amount of 20 mg/kg every 2 weeks or every 4 weeks, or in an amount of 1500 mg every 2 weeks, 3 weeks, or 4 weeks. In embodiments of any of the above aspects, durvalumab is administered at a dose of 10 mg/kg Q2W or at a dose of 1500 mg Q4W. In an embodiment of any of the above aspects, durvalumab is administered via intravenous infusion.

In various embodiments of any of the above aspects, the subject is identified as responsive to treatment with an anti-PD-L1 antibody, or with an anti-PD-L1 antibody in combination with an anti-CTLA4 antibody, or an antigen binding fragment thereof and achieves disease control (DC) as described herein. In various embodiments of any of the above aspects, PD-L1 expression on bladder cancer cells and tissue is detected using immunohistochemistry (e.g., on cancer cells that are formalin fixed and paraffin embedded). In various embodiments of any of the above aspects, the methods result in an increase in overall survival (OS), (e.g., an increase of weeks, months, or years) compared to the administration of standard-of-care therapies, e.g., platinum-based therapies. In particular, the increase in survival is more than about 4-6 weeks, 1-2 months, 3-4 months, 5-7 months, 6-8 months, or 9-12 months, or longer. In various embodiments of any of the above aspects, the administration of durvalumab is repeated at or about every 2 weeks or at or about every 4 weeks.

In various embodiments of any of the above aspects, the administration of tremelimumab or an antigen-binding fragment thereof, when used in combination with durvalumab, is repeated about every 4 weeks. In various embodiments of any of the above aspects, the administration of tremelimumab or an antigen-binding fragment thereof is repeated about every 12 weeks. In various embodiments of any of the above aspects, the administration of tremelimumab or an antigen-binding fragment thereof is administered about every 4 weeks for seven administrations and then every 12 weeks. In various embodiments of any of the above aspects, the administration of durvalumab and/or tremelimumab or antigen binding fragments thereof to a subject in need thereof is by intravenous infusion. In various embodiments of any of the above aspects, durvalumab and an additional anti-cancer or immunotherapeutic agent, e.g., tremelimumab are administered concurrently or at different times. In various embodiments of any of the above aspects, durvalumab and tremelimumab are administered 12, 24, 26, 48, or 72 hours apart; 1, 2, or 3 weeks apart, or between 1, 2, and 3 months apart. In various embodiments of any of the above aspects, a bladder cancer tumor expresses low (reduced) or undetectable (negligible or negative) levels of PD-L1. In various embodiments of any of the above aspects, the bladder cancer, e.g., UC, is low or low/neg for PD-L1 expression when fewer than 25% of cancer or tumor cells in a population of cancer or tumor cells express PD-L1 or show positive staining for PD-L1 when a PD-L1 detection agent (e.g., a detectably labeled anti-PD-L1antibody) is used. In various embodiments of the above aspects, the described treatment methods result in an increase in overall survival, objective response rate, progression free survival, or a complete response in the subject. In various embodiments of the above aspects, the median time to treatment response by the subject ranges from about 1 month to 8 months. In various embodiments of the above aspects, the duration of response by the subject is at least 6 months, 9 months, 12 months, or longer. In embodiments of the above methods, a treatment response (e.g., overall survival (OS)) is detected in over 60% (e.g., 68%) of subjects having high PD-L1 expression on bladder cancer or tumor cells/tissue at about 6 months; and a treatment response (e.g., OS) is detected in over 40% (e.g., 45%) of subjects having low/negative PD-L1expression on bladder cancer or tumor cells/tissue at about 6 months. In embodiments of the above methods, a treatment response (e.g., overall survival (OS)) is detected in over 60% (e.g., 65%) of subjects having high PD-L1 expression on bladder cancer or tumor cells/tissue at about 9 months; and a treatment response (e.g., OS) is detected in over 35% (e.g., 36%) of subjects having low/negative PD-L1 expression on bladder cancer or tumor cells/tissue at about 9 months. In embodiments of the above methods, a treatment response (e.g., overall survival (OS)) is detected in about 60% of subjects having high PD-L1 expression on bladder cancer or tumor cells/tissue at about 12 months; and a treatment response (e.g., OS) is detected in about 35% of subjects having low/negative PD-L1 expression on bladder cancer or tumor cells/tissue at about 12 months. In other embodiments of the above methods, about 60% of subjects with UC have an overall survival (OS) response at about 6 months; over 55% (e.g., 56%) of subjects with UC have an OS response at about 9 months; and over 50% (e.g., 52%) of subjects with UC have an OS response at about 12 months.

Other features and advantages of the invention will be apparent from the detailed description, and the claims.

### BRIEF DESCRIPTION OF THE DRAWINGS/FIGURES

FIG. 1 is a summary of a patient population in the clinical study (Study 1108) described herein. In FIG. 1, 1L=first line; 2L =second line or greater; DCO=data cut-off date; UC=urothelial carcinoma. DCO date for non-UC patients=29 Apr. 2016; DCO date for patients in the UC cohort=24 Jul. 2016. 2L+ post-platinum patients had progressed while on or after a platinum-based therapy, including those patients who progressed within 12 months of receiving therapy in a neo-adjuvant/adjuvant setting.

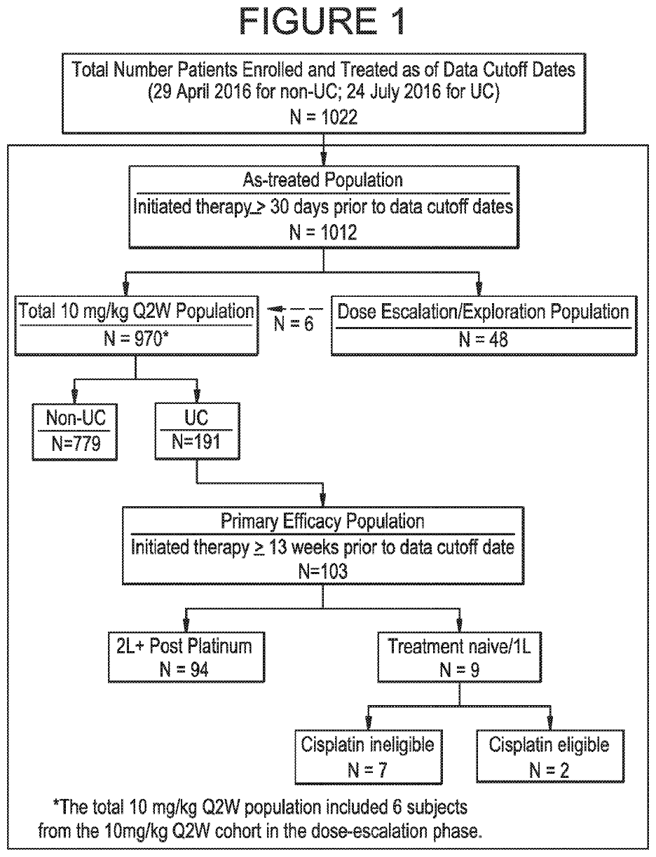

FIG. 2 depicts a Kaplan-Meier analysis of duration of response as determined by blinded independent central review (BICR) of the UC cohort in Study 1108 (Primary Efficacy Population: treated patients with an opportunity for ≥13 weeks of follow-up), as described in Example 2 herein. In the figure, CI=Confidence Interval; NR=Not Reached; NE=Not Estimable; Prob.=Probability; Resp=Response; UC=Urothelial Carcinoma.

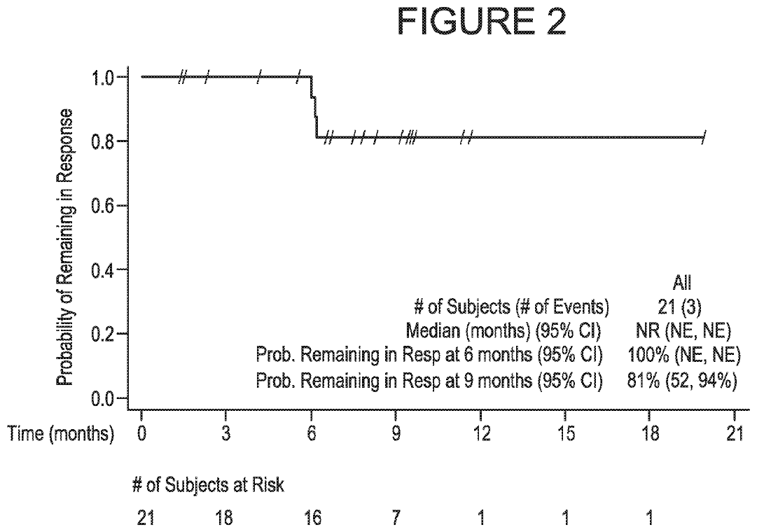

FIG. 3 depicts a bar graph showing time to response and duration of response as determined by blinded independent central review (BICR) based on PD-L1 status of the patients' tumors in the UC cohort of Study 1108 (Primary Efficacy Population: treated patients with an opportunity for ≥13 weeks of follow-up) as described in Example 2 herein. In the figure, Neg=Negative; PD-L1=Programmed Cell Death Ligand-1; UC=Urothelial Carcinoma.

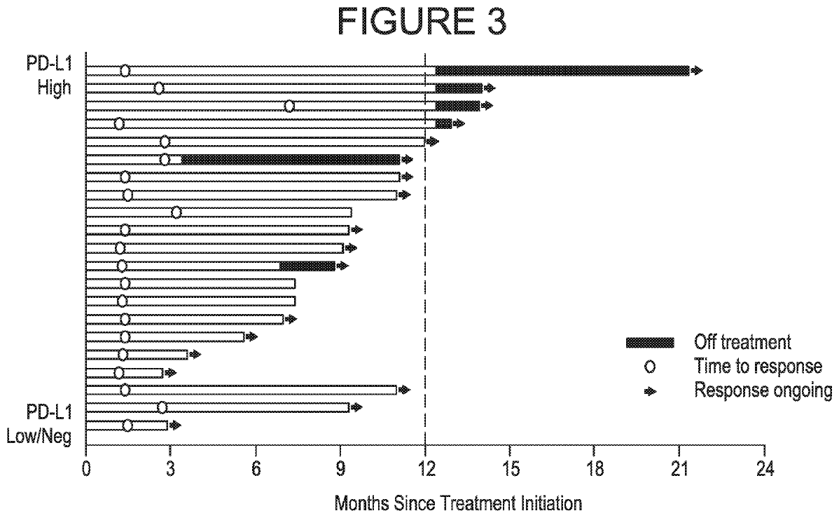

FIG. 4 presents a Kaplan-Meier plot showing an estimate of overall survival (OS) in the UC cohort in Study 1108 (Primary Efficacy Population: treated patients with an opportunity for ≥13 weeks of follow-up) as described in Example 2 herein. The “All” column (c) includes 3 subjects who had unknown PD-L1 status and who are not included in either the (a) PD-L1-high or the (b) PD-L1-low/neg subgroups. In the figure, CI=Confidence Interval; NE=Not Estimable; Neg=Negative; NR=Not Reached; OS=Overall Survival; UC=Urothelial Carcinoma.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “anti-PD-L1 antibody” is meant an antibody that selectively binds a PD-L1 polypeptide. Exemplary anti-PD-L1 antibodies are described for example at WO 2011/066389, which is herein incorporated by reference. Durvalumab (MED14736) is an exemplary anti-PD-L1antibody that is suitable for the methods described herein. The sequences are provided in the sequence listing herein (e.g., SEQ ID NOs. 3-10).

By “low/neg for PD-L1” is meant that a cell or population of cells express(es) significantly reduced, low, or undetectable (e.g., negative or negligible) levels of PD-L1 relative to a PD-L1-positive cell or population of cells. In an embodiment, PD-L1 expression is on the surface of a tumor, cancer, or immune cell. In an embodiment, expression is low/negligible, when levels of PD-L1 are reduced by at least about 5%, 10%, 25%, 30%, 40%, 50%, 60%, 70%, 80%, 90% or more relative to a PD-L1high cell or population of cells. In another embodiment, expression is low or negligeable (e.g., PD-L-1low/neg) when fewer than about 5%, 10%, 25%, 30%, 40%, 50%, 60%, 70%, 80%, 90% or more of the cells in a population (e.g., bladder cancer/tumor cells, such as UC cells) express detectable levels of PD-L1 protein or polynucleotide. In a particular embodiment, such as in the context of immunohistochemistry, “low PD-L1” (or PD-L1low) or “low/neg PD-L1” (or PD-L1-low/neg) means that less than about 25% of cells in a cancer sample exhibit staining for PD-L1. In a particular embodiment, such as in the context of immunohistochemistry, “low PD-L1” (or PD-L1-low) or “low/neg PD-L1” (or PD-L1low/neg) means that 25% or fewer cells in a cancer sample exhibit staining for PD-L1. In another particular embodiment, reduced, low, or low/neg levels of PD-L1 expression refer to fewer than 25% of the cells or tissue derived from a subject's bladder cancer, tumor, or tissue, such as a UC tumor, detected as expressing PD-L1. In a particular embodiment, high levels of PD-L1 expression refer to greater than 25%, 30%, 40%, 50%, 60%, 70%, 80%, 90% or more of the cells or tissue derived from a subject's bladder cancer, tumor, or tissue, e.g., a UC tumor, detected as expressing PD-L1. It will be appreciated by the skilled practitioner that cut-off values for high and low, or low/neg levels of expression of PD-L1 on bladder cancer or tumor cells and/or tissue may be determined by different detection and assay procedures known and practiced in the art and may vary depending on the detection system employed. In an embodiment, immunohistochemistry or staining is employed, along with cell sorting analysis, e.g., fluorescent activated cell sorting (FACS).

By “PD-L1 polypeptide” is meant a polypeptide or fragment thereof having at least about 85%, or greater, amino acid identity to NCBI Accession No. NP_001254635 (SEQ ID NO: 1, below) and haying PD-1 and CD80 binding activity. PD-L1 may be used interchangeably with the term “B7-H1”).

```
    1 MRIFAVFIFM TYWHLLNAPY NKINQRILVV
        DPVTSEHELT CQAEGYPKAE VIWTSSDHQV
  61 LSGKTTTTNS KREEKLFNVT STLRINTTTN
        EIFYCTFRRL DPEENHTAEL VIPELPLAHP
121 PNERTHLVIL GAILLCLGVA LTFIFRLRKG
        RMMDVKKCGI QDTNSKKQSD THLEET
```

By “PD-L1 nucleic acid molecule” is meant a polynucleotide encoding a PD-L1 polypeptide. An exemplary PD-L1 nucleic acid molecule sequence is provided at NCBI Accession No. NM_001267706 and in SEQ ID NO: 2, below)

```
      1 ggcgcaacgc tgagcagctg gcgcgtcccg
          cgcggcccca gttctgcgca gcttcccgag
    61 gctccgcacc agccgcgctt ctgtccgcct
          gcagggcatt ccagaaagat gaggatattt
  121 gctgtcttta tattcatgac ctactggcat
          ttgctgaacg ccccatacaa caaaatcaac
  181 caaagaattt tggttgtgga tocagtcacc
          tctgaacatg aactgacatg tcaggctgag
  241 ggctacccca aggccgaagt catctggaca
          agcagtgacc atcaagtcct gagtggtaag
  301 accaccacca ccaattccaa gagagaggag
          aagcttttca atgtgaccag cacactgaga
  361 atcaacacaa caactaatga gattttctac
          tgcactttta ggagattaga tcctgaggaa
  421 aaccatacag ctgaattggt catcccagaa
          ctacctctgg cacatcctcc aaatgaaagg
  481 actcacttgg taattctggg agccatctta
          ttatgccttg gtgtagcact gacattcatc
  541 ttccgtttaa gaaaagggag aatgatggat
          gtgaaaaaat gtggcatcca agatacaaac
  601 tcaaagaagc aaagtgatac acatttggag
          gagacgtaat ccagcattgg aacttctgat
  661 cttcaagcag ggattctcaa cctgtggttt
          aggggttcat cggggctgag cgtgacaaga
  721 ggaaggaatg ggcccgtggg atgcaggcaa
          tgtgggactt aaaaggccca agcactgaaa
  781 atggaacctg gcgaaagcag aggaggagaa
          tgaagaaaga tggagtcaaa cagggagcct
  841 ggagggagac cttgatactt tcaaatgcct
          gaggggctca tcgacgcctg tgacagggag
  901 aaaggatact tctgaacaag gagcctccaa
          gcaaatcatc cattgctcat cctaggaaga
  961 cgggttgaga atccctaatt tgagggtcag
          ttcctgcaga agtgcccttt gcctccactc
1021 aatgcctcaa tttgttttct gcatgactga
          gagtctcagt gttggaacgg gacagtattt
1081 atgtatgagt ttttcctatt tattttgagt
          ctgtgaggtc ttcttgtcat gtgagtgtgg
1141 ttgtgaatga tttcttttga agatatattg
          tagtagatgt tacaattttg togccaaact
1201 aaacttgctg cttaatgatt tgctcacatc
          tagtaaaaca tggagtattt gtaaggtgct
1261 tggtctcctc tataactaca agtatacatt
          ggaagcataa agatcaaacc gttggttgca
1321 taggatgtca cctttattta acccattaat
          actctggttg acctaatctt attctcagac
1381 ctcaagtgtc tgtgcagtat ctgttccatt
          taaatatcag ctttacaatt atgtggtagc
1441 ctacacacat aatctcattt catcgctgta
          accaccctgt tgtgataacc actattattt
1501 tacccatcgt acagctgagg aagcaaacag
          attaagtaac ttgcccaaac cagtaaatag
1561 cagacctcag actgccaccc actgtccttt
          tataatacaa tttacagcta tattttactt
1621 taagcaattc ttttattcaa aaaccattta
          ttaagtgccc ttgcaatatc aatcgctgtg
1681 ccaggcattg aatctacaga tgtgagcaag
          acaaagtacc tgtcctcaag gagctcatag
1741 tataatgagg agattaacaa gaaaatgtat
          tattacaatt tagtccagtg tcatagcata
1801 aggatgatgc gaggggaaaa cccgagcagt
          gttgccaaga ggaggaaata ggccaatgtg
1861 gtctgggacg gttggatata cttaaacatc
          ttaataatca gagtaatttt catttacaaa
1921 gagaggtcgg tacttaaaat aaccctgaaa
          aataacactg gaattccttt tctagcatta
1981 tatttattcc tgatttgcct ttgccatata
          atctaatgct tgtttatata gtgtctggta
2041 ttgtttaaca gttctgtctt ttctatttaa
          atgccactaa attttaaatt catacctttc
2101 catgattcaa aattcaaaag atcccatggg
          agatggttgg aaaatctcca cttcatcctc
2161 caagccattc aagtttcctt tccagaagca
          actgctactg cctttcattc atatgttctt
2221 ctaaagatag tctacatttg gaaatgtatg
          ttaaaagcac gtatttttaa aatttttttc
2281 ctaaatagta acacattgta tgtctgctgt
          gtactttgct atttttattt attttagtgt
2341 ttcttatata gcagatggaa tgaatttgaa
          gttcccaggg ctgaggatcc atgcottctt
2401 tgtttctaag ttatctttcc catagctttt
          cattatcttt catatgatcc agtatatgtt
2461 aaatatgtcc tacatataca tttagacaac
          caccatttgt taagtatttg ctctaggaca
2521 gagtttggat ttgtttatgt ttgctcaaaa
          ggagacccat gggctctcca gggtgcactg
2581 agtcaatcta gtcctaaaaa gcaatcttat
          tattaactct gtatgacaga atcatgtctg
2641 gaacttttgt tttctgcttt ctgtcaagta
          taaacttcac tttgatgctg tacttgcaaa
2701 atcacatttt ctttctggaa attccggcag
          tgtaccttga ctgctagcta ccctgtgcca
2761 gaaaagcctc attcgttgtg cttgaaccct
          tgaatgccac cagctgtcat cactacacag
2821 ccctcctaag aggcttcctg gaggtttcga
          gattcagatg ccctgggaga tcccagagtt
2881 tcctttccct cttggccata ttctggtgtc
          aatgacaagg agtaccttgg ctttgccaca
2941 tgtcaaggct gaagaaacag tgtctccaac
          agagctcctt gtgttatctg tttgtacatg
3001 tgcatttgta cagtaattgg tgtgacagtg
          ttctttgtgt gaattacagg caagaattgt
3061 ggctgagcaa ggcacatagt ctactcagtc
          tattcctaag tcctaactcc tccttgtggt
3121 gttggatttg taaggcactt tatccctttt
          gtctcatgtt tcatogtaaa tggcataggc
3181 agagatgata cctaattctg catttgattg
          tcactttttg tacctgcatt aatttaataa
3241 aatattctta tttattttgt tacttggtac
          accagcatgt ccattttctt gtttattttg
3301 tgtttaataa aatgttcagt ttaacatccc
          agtggagaaa gttaaaaaa
```

By “an anti-CTLA4 antibody” is meant an antibody that selectively binds a CTLA4 polypeptide. Exemplary anti- CTLA4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody. Tremelimumab sequences arc provided in the sequence listing below.

The term “antibody” as used herein refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as antigen binding fragment (Fab), F(ab′)₂, F(ab′), variable domain fragment (Fv), single chain antibodies; single chain variable fragments (scFv), Fd, dAb, single chain antibodies, disulfide-linked Fvs (sdFv), intrabodies and other antibody fragments that retain antigen-binding function, i.e., the ability to bind PD-L1specifically. Typically, such fragments comprise an antigen-binding domain, e.g., a PD-L1-binding domain, or contain one or more complementary determining regions (CDRs), e.g., CDR1, CDR2, CDR3, from the light and heavy chain variable regions that specifically hind antigen, e.g., PD-L1 polypeptide antigen.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}); however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain. The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

By “biologic sample” is meant any tissue, cell, fluid, or other material derived from an organism. In one embodiment, a biological sample is a bladder cancer or UC tumor biopsy sample.

A “biomarker” or “marker” as used herein generally refers to a protein, nucleic acid molecule, clinical indicator, or other analyte that is associated with a disease. In one embodiment, a marker is differentially present in a biological sample obtained from a subject having a disease (e.g., bladder cancer) relative to the level present in a control sample or reference.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining”, “assessing”, “assaying”, “measuring” and “detecting”, and “identifying” refer to both quantitative and qualitative determinations, and as such, the term “determining” is used interchangeably herein with “assaying,” “measuring,” and the like. Where a quantitative determination is intended, the phrase “determining an amount” of an analyte, substance, protein, and the like is used. Where a qualitative and/or quantitative determination is intended, the phrase “determining a level” of an analyte or “detecting” an analyte is used.

By “disease” is meant any condition or disorder that damages, interferes with or dysregulates the normal function of a cell, tissue, or organ. In a disease such as cancer (e.g., bladder cancer) the normal function of a cell tissue or organ is subverted to enable immune evasion and/or escape. As noted supra, bladder cancer includes cancer of any portion of the bladder, e.g., muscle or epithelium, as well as ductal structures such as the ureter, or the urethra, or the urachus. A bladder cancer may be muscle-invasive or non-muscle-invasive, based on invasion by cancer cells of the muscularis propria.

The terms “isolated,” “purified” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified, as used herein, if it is substantially free of cellular material, viral material, or culture medium when produced by recombinant DNA techniques, or chemical precursors, or other chemicals when chemically synthesized. Purity and homogeneity are typically determined using analytical chemistry techniques, for example, polyacrylamide gel electrophoresis, high performance liquid chromatography (HPLC), mass spectrometry analysis, etc. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reference” is meant a standard of comparison.

By “responsive” in the context of therapy is meant susceptible to treatment.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶M⁻¹, or more preferably higher than 10⁸M⁻¹. If necessary, non-specific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques.

By “subject” is meant a mammal, including, but not limited to, a human, such as a human patient, a non-human primate, or a non-human mammal, such as a bovine, equine, canine, ovine, or feline animal.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, diminishing, lessening, alleviating, abrogating, or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from its context, the term “or” as used herein is understood to be inclusive. Unless specifically stated or obvious from context, the terms “a”, “an”, and “the” as used herein are understood to be singular or plural.

Unless specifically stated or obvious from context, as used herein, the term “about” is understood as within a range of normal tolerance in the art, for example within 2 standard deviations of the mean. The term “about” is understood to refer to within 5%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

As described below, the present invention features methods of treating bladder cancer, e.g., urothelial carcinoma (UC), in a subject having bladder cancer and/or in a subject who is identified as having a cancer or tumor which expresses different levels of PD-L1, such as a PD-L1low/negligible tumor, or a PD-L1 high tumor, with an anti-PD-L1 antibody, in particular, durvalumab. The bladder cancer treatment methods described herein involve administering the anti-PD-L1 antibody as a first line cancer therapy in an effective amount to treat the subject's bladder cancer. The treatment methods described herein may also involve administering an effective amount of the anti-PD-L1 antibody to a subject having bladder cancer after the subject has been treated with a first-line cancer therapy, such as a standard-of-care (SoC) therapy, e.g., with a platinum-containing drug, but has progressed or relapsed following the first-line SoC therapy. In a particular embodiment, the anti-PD-L1 antibody is durvalumab (MEDI4736). In embodiments, the anti-PD-L1 antibody is co-administered, either simultaneously or sequentially, with an effective amount of another cancer therapeutic or immunotherapeutic agent, e.g., an anti-CTLA4 antibody, such as tremelimumab, or an antigen binding fragment thereof.

### PD-L1

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients.

Two T cell modulatory pathways that are of importance in immune cell function signal through the programmed death ligand 1 (PD-L1, also known as B7H-1 or CD274) protein and cytotoxic T lymphocyte antigen-4 (CTLA-4, CD152) protein.

PD-L1 is part of a complex system of immunomodulating receptors and ligands that are involved in controlling T cell activation. The PD-L1 protein is a member of the B7 family of ligands that inhibit T-cell activity through binding to the PD-1 receptor and to CD80). In normal tissues, PD-L1 is expressed on T cells, B cells, dendritic cells, macrophages, mesenchymal stem cells, bone marrow-derived mast cells, as well as on various non-hematopoietic cells. Its normal function is to regulate the balance between T-cell activation and tolerance through interaction with its two receptor proteins: ‘programmed death 1’ (also known as PD-1 or CD279) and CD80 (also known as B7-1 or B7.1).

PD-L1 is also expressed by tumors and acts at multiple sites to help tumors evade detection and elimination by the host immune system. PD-L1 is expressed in a broad range of cancers with a high frequency. Expression of PD-L1 on both tumor cells (TC) and tumor-infiltrating immune cells (IC) is induced by inflammatory signals that are typically associated with an adaptive immune response (e.g., IFNy production). The binding of PD-L1 to PD-1 on activated T cells delivers an inhibitory signal to the T cells, thereby protecting the tumor from immune elimination. PD-L1 may also inhibit T cell activity through binding to CD80, although the exact mechanism is under investigation. In some cancers, expression of PD-L1 has been associated with reduced survival and unfavorable prognosis. Antibodies that block the interaction between PD-L1 and its receptors are able to relieve PD-L1 -dependent immunosuppressive effects and enhance the cytotoxic activity of antitumor T cells in vitro. In addition, in vivo studies have shown that durvalumab inhibits tumor growth in xenograft models via a T cell-dependent mechanism. Without being bound by theory, durvalumab in the present methods stimulates a bladder cancer subject's anti-tumor immune response by binding to PD-L1and shifting the balance toward an anti-tumor response.

### CTLA4

In contrast to PD-L1, cytotoxic T-lymphocyte-associated antigen-4 (CTLA-4) is constitutively expressed by regulatory T cells and is up-regulated on activated T cells. CTLA4 serves as a co-inhibitor to keep T cell responses in check following CD28-mediated T cell activation. Following T cell receptor (TCR) engagement, CTLA4 is believed to regulate the amplitude of the early activation of naïve and memory T cells and to be part of a central inhibitory pathway that affects both antitumor immunity and autoimmunity. CTLA4 is expressed primarily on T cells, and the expression of its ligands CD80 (B7.1) and CD86 (B7.2) is largely restricted to antigen-presenting cells, T cells, and other immune mediating cells. Binding of CTLA-4 to CD80 or CD86 on tumor-infiltrating immune cells (IC) leads to inhibition of T-cell activation. Antagonistic anti-CTLA4 antibodies that block the CTLA4 signaling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma, but did not significantly increase the overall survival of patients compared to the standard of care (temozolomide or dacarbazine) at that time.

### Anti-PD-L1 Antibodies

Antibodies that specifically bind and inhibit PD-L1 activity (e.g., binding to PD-1 and/or CD80) are useful for the treatment of bladder cancer (e.g., UC).

Durvalumab (MEDI4736), an exemplary anti-PD-L1 antibody, is a human monoclonal antibody (mAb), (immunoglobulin G1 (IgG1) kappa), that is genetically engineered to reduce antibody-dependent cell-mediated cytotoxicity (ADCD). Durvalumab binds to PD-L1 and blocks its interaction with PD-1 receptors on T cells and cluster of differentiation (CD80, B7.1) receptors on tumor-infiltrating immune cells (IC). Durvalumab can relieve PD-L1-mediated suppression of human T-cell activation in vitro by antagonizing the inhibitory effect of PD-L1 on primary human T cells, resulting in their restored proliferation and release of interferon gamma (IFNγ), and can inhibit tumor growth in a xenograft model via a T-cell dependent mechanism.

Information regarding durvalumab (or fragments thereof) for use in the methods provided herein can be found in U.S. Pat. No. 8,779,108, the disclosure of which is incorporated herein by reference in its entirety. The fragment crystallizable (Fc) domain of durvalumab contains a triple mutation in the constant domain of the IgG1 heavy chain that reduces binding to the complement component Clq and the Fcγ receptors responsible for mediating antibody-dependent cell-mediated cytotoxicity (ADCC).

Durvalumab (MEDI4736), and antigen-binding fragments thereof, for use in the methods provided herein comprises a heavy chain and a light chain or a heavy chain variable region and a light chain variable region. In a specific aspect, durvalumab or an antigen-binding fragment thereof for use in the methods provided herein comprises a light chain variable region comprising the amino acid sequence of SEQ ID NO: 3 and a heavy chain variable region comprising the amino acid sequence of SEQ ID NO: 4. In a specific aspect, durvalumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 5-7, and wherein the light chain variable region comprises the Kabat-defined CDR1, CDR2, and CDR3 sequences of SEQ ID NOs: 8-10. Those of ordinary skill in the art would easily be able to identify Chothia-defined, Abm-defined, Kabat-defined, or other CDR definitions known to those of ordinary skill in the art. In a specific aspect, durvalumab or an antigen-binding fragment thereof for use in the methods provided herein comprises the variable heavy chain and variable light chain CDR sequences of the 2.14H9OPT antibody as disclosed in WO 2011/066389 A1, which is herein incorporated by reference in its entirety.

The bladder cancer treatment methods described herein are especially effective for subjects having bladder cancer, namely, UC. The methods are also effective for treating subjects whose bladder cancer or tumor cells or tissue have been identified as expressing low levels of PD-L1 (PD-L1-low) or negative/low levels of PD-L1 (PD-L1-low/neg), including UC cells or tissue. In addition, the methods herein are effective for treating bladder cancer, e.g., UC, in subjects having bladder cancer (e.g., UC) tumors expressing high levels of PD-L1 (e.g., PD-L1high tumors).

### Presently Available Therapies For Advanced or Metastatic Urothelial Carcinomas (UC) and Their Related Disadvantages

### Initial Therapy for Locally Advanced Disease

Locally advanced UC is usually treated by radical cystectomy and neoadjuvant and adjuvant chemotherapy. Systemic combination chemotherapy with a platinum agent is the regimen of choice in both the neoadjuvant and adjuvant setting. Patients who have recurrence of disease more than 12 months after neoadjuvant or adjuvant therapy may be retreated with a platinum-based therapy as the first-line (1L) regimen, as described below, However, approximately 25% of patients have recurrence of disease within 1 year of therapy. These patients are not eligible for standard platinum-based therapy, and have limited treatment options.

### First-Line Platinum-Based Therapy for Locally Advanced or Metastatic Disease

For patients with locally advanced or metastatic UC, systemic cisplatin-containing combination chemotherapy, introduced nearly 30 years ago, is currently the standard of care (SoC). First-line (1L) chemotherapy includes cisplatin/gemcitabine or methotrexate, vinblastine, ADRIAMYCIN™ (doxorubicin), and cisplatin (MVAC). These treatment regimens have yielded objective response rates (ORRs) of 46% to 60%, median progression-free survival (PFS) of 7 to 8 months, and median overall survival (OS) of 13 to 15 months in the 1L therapeutic regimen. However, approximately half of patients are unfit for cisplatin-containing chemotherapy due to a poor performance status, impaired renal function, or co-morbidity; such patients are generally given palliative therapy with a carboplatin-based regimen or single-agent taxane or gemcitabine. These treatments are less effective than cisplatin-based chemotherapy, yielding objective response rates (ORRs) of approximately 30% to 40%; median PFS of approximately 3 to 6 months; and median OS that rarely exceeds 10 months.

### Post-Platinum Therapy

Despite clinical benefits in the 1L setting, disease progression invariably occurs in patients after they discontinue chemotherapy, even in patients who initially respond to chemotherapy. Approximately 6,500 patients progress to second-line (2L) therapy for locally advanced or metastatic UC in the United States annually.

Currently, there is no available therapy or standard therapy in the United States for patients who have progressed following 1L therapy, or who have relapsed within 1 year after neoadjuvant or adjuvant therapy. In Europe, vinflunine was approved for 2L therapy based on a randomized Phase 3 trial comparing vinflunine+best supportive care (BSC) versus BSC alone in patients with UC who progressed following 1L platinum-containing chemotherapy. However, this trial did not meet its primary OS endpoint. A post-hoc multivariate Cox analysis adjusting for prognostic factors showed a 23% reduction in the risk of death of patients who had received vinflunine+BSC compared with those who had received BSC alone (hazard ratio [HR]=0.77 [95% CI: 0.61, 0.98]; p=0.036). In the eligible population, the median OS was longer for patients receiving vinflunine+BSC treatment than for those receiving BSC treatment (6.9 versus 4.3 months, respectively; HR=0.78 [95% CI: 0.61, 0.99]; p=0.040). Improved ORR (8.6% versus 0%; 95% CI: 5.0%, =13.7%; p=0.006) and median PFS (3.0 versus 1.5 months; p=0.001) were observed in the evaluable patient population. In the trial, the median duration of response (DoR) for vinflunine therapy was 7.4 months.

In the United States, post-platinum therapies include off-label use of a taxane (docetaxel, paclitaxel, nab-paclitaxel) or a combination of paclitaxel with gemcitabine. Evidence supporting the use of these agents is primarily from small, uncontrolled Phase 2 studies, with highly variable results that are dependent on patient selection. An analysis of frequently-used second line (2L) treatment with single-agent cytotoxic chemotherapies demonstrated ORRs of approximately 10% and no improvement in OS. A meta-analysis of 2L single-agent and doublet chemotherapies, including cisplatin or carboplatin, showed an improvement in ORR (14.2% versus 31.9%, respectively) with doublet chemotherapy, and a modest difference in median PFS (2.7 versus 4.1 months, respectively) or median OS (7.0 versus 8.5 months), respectively. Pooled data from ten Phase 2 trials evaluating 2L chemotherapy, biologics, and combination therapy with chemotherapy and biologic agents showed an OS rate at 12 months of 20% (95% CI: 17, 24).

These chemotherapies are palliative and have significant adverse reactions that can lead to intolerance to treatment. Reported Grade 3 or 4 toxicities have included, but are not limited to, neutropenia, anemia, fatigue, constipation, and thrombocytopenia. Locally advanced or metastatic UC in patients who had previously received a platinum-based therapy is a serious and life-threatening condition, for which new treatments and therapeutic regimens are seriously needed. The present methods provide such needed treatments and therapeutic regimens for patients having UC, advanced UC, or metastatic UC.

### Anti-PD-L1 (Durvalumab) Monotherapy or Combination Therapy for Bladder Cancer, e.g., UC, Treatment

The methods described herein afford medical and clinical benefits associated with the administration of an anti-PD-L1 antibody, such as durvalumab, to a subject having bladder cancer, e.g., UC, and who is in need of treatment, in an effective amount to treat the bladder cancer, e.g., UC, in the subject. The therapeutic benefits of an anti-PD-L1 antibody, in particular, durvalumab, as a monotherapy or first line (1L) treatment for a bladder cancer such as UC are described herein and demonstrated in the Examples.

There is currently neither an available therapy nor a standard therapy in the United States for patients with locally advanced or metastatic urothelial carcinoma (UC) who have progressed following first-line (1L) therapy or who have relapsed within 1 year after neoadjuvant or adjuvant therapy, e.g., involving treatment with a platinum-containing chemotherapeutic drug or drug regimen. Post-platinum chemotherapies are generally palliative with limited clinical effectiveness and significant adverse reactions. Locally advanced or metastatic UC is a life-threatening disease, with a large unmet need for new treatment options in patients who have progressed during or after undergoing treatment involving a platinum-containing chemotherapeutic drug regimen.

Accordingly, to address this need, the present methods provide effective therapeutic benefit related to the use of an anti-PD-L1 antibody, in particular, durvalumab, for treating a subject having bladder cancer, such as UC, and for treating a subject having bladder cancer, e.g., UC, whose cancer has progressed, or who has relapsed within a year after a first line (1L) anti-cancer treatment, such as with platinum-containing chemotherapeutic drug or drug regimen.

In embodiments of all of the described methods of the invention, the subject undergoing treatment, or to be treated, in accordance with the present methods is identified as having a bladder cancer, in particular, UC, with various levels of PD-L1 expression. In some embodiments, the cancer or tumor cells or tissue of the subject with bladder cancer, in particular, UC, express negative-to-low levels of PD-L1 (PD-L1 -low/neg), or high levels of PD-L1 (PD-L1 -high). According to the present invention, the methods of treatment involving an anti-PD-L1antibody, and in particular, durvalumab, for the treatment of bladder cancer, especially UC, embrace administering to a subject in need an effective amount of the anti-PD-L1 antibody, particularly, durvalumab, to treat the subject's bladder cancer, e.g., UC, when the cancer or tumor is identified as PD-L1-low/neg, or PD-L1-high for PD-L1 expression. In an embodiment, a cancer or tumor cell or tissue is considered to be PD-L1low/neg when PD-L1 expression is less than 25% relative to a suitable control, for example, as measured by immunohistochemistry or staining assay. In an embodiment, a cancer or tumor cell or tissue is considered to be PD-L1-high when PD-L1 expression is greater than 25% relative to a suitable control, for example, as measured by immunohistochemistry or staining assay. As will be appreciated by the skilled practitioner, the cutoff values for expression of PD-L1 on cancer or tumor cells and tissues determined to express PD-L1 at PD-L1-low/neg or PD-L1-high levels relative to control levels depends on the type of assay or assays employed to determine PD-L1 expression; such assays are readily able to be practiced by those having skill in the art.

In embodiments of all of the above methods, the anti-PD-L1 antibody is durvalumab administered in an effective amount (or dose) of 1 mg/kg to 50 mg/kg, or about 4 mg/kg to 5 mg/kg, or 5 mg/kg to 10 mg/kg, or 10 mg/kg to 50 mg/kg, or 10 mg/kg to 30 mg/kg, or 10 mg/kg to 20 mg/kg, or 10 mg/kg, or 20 mg/kg, or 1500 mg, every week, every two weeks, (Q2W), every three weeks (Q3W), every four weeks (Q4W), every five weeks (Q5W), every six weeks (Q6W), every seven weeks (Q7W), every eight weeks (Q8W) up to every six months, or longer. In a particular embodiment, durvalumab is administered to a subject with bladder cancer such as UC in an effective amount of 10 mg/kg Q2W as a bladder cancer (or UC) therapeutic. In a particular embodiment, durvalumab is administered to a subject with bladder cancer such as UC in an effective amount of 10 mg/kg Q4W as a bladder cancer (or UC) therapeutic. In a particular embodiment, durvalumab is administered to a subject with bladder cancer such as UC in an effective amount of 20 mg/kg Q2W as a bladder cancer (or UC) therapeutic. In a particular embodiment, durvalumab is administered to a subject with bladder cancer such as UC in an effective amount of 20 mg/kg Q4W as a bladder cancer (or UC) therapeutic. In a particular embodiment, durvalumab is administered to a subject with bladder cancer such as UC in an effective amount of 1.5 g Q2W as a bladder cancer (or UC) therapeutic. In a particular embodiment, durvalumab is administered to a subject with bladder cancer such as UC in an effective amount of 1.5 g Q4W as a bladder cancer (or UC) therapeutic. In another embodiment, the anti-PD-L1 antibody, e.g., durvalumab, is administered intravenously, such as via intravenous (IV) infusion. In an embodiment, the IV infusion delivers the anti-PD-L1 antibody, e.g., durvalumab, over a predetermined time period, such as over 10, 20, 30, 40, 50, 60, 70, 80, or 90 minutes. In a particular embodiment, the IV infusion delivers the dose of anti-PD-L1antibody, e.g., durvalumab, over 60 minutes. In related embodiments, the anti-PD-L1 antibody, e.g., durvalumab, is administered until an endpoint is attained, e.g., an overall or complete response, or overall survival, by the subject, disease progression, or unacceptable toxicity. Such endpoints are able to be determined by a skilled medical practitioner or clinician.

In another embodiment, the anti-PD-L1 antibody (e.g., durvalumab) is administered in conjunction with another therapeutic, immunotherapeutic, or chemotherapeutic agent, such as an anti-CTLA4 antibody (e.g., tremelimumab). In some embodiments, a standard of care drug, such as a platinum chemotherapeutic, a platinum-containing chemotherapeutic and the like are also administered with the anti-PD-L1 and/or the anti-CTLA4 antibodies or antigen binding fragments thereof. In such a combination therapy, the antibodies may be administered separately or together, at the same time or at different times. In addition, the standard of care drug may be administered at the same time as, or at different times from, the administration of the anti-PD-L1antibody and/or the anti-CTLA4 antibody.

### Anti-PD-L1 and Anti-CTLA4 (Tremelimumab) Treatment

Subjects suffering from bladder cancer (e.g., UC) may be tested for PD-L1polynucleotide or polypeptide expression in the course of selecting a treatment method. Subjects identified as having tumors that express negligible or low PD-L1 (e.g., as defined by Ct or IHC-M score), or as having reduced (low) or undetectable levels of PD-L1 relative to a reference level, are identified as responsive to treatment with an anti-PD-L1 antibody, such as durvalumab, or with a combination of an anti-PD-L1 antibody and an anti-CTLA4 antibody such as tremelimumab. Such subjects are administered an anti-PD-L1 antibody, such as durvalumab, or an antigen-binding fragment thereof, in combination with tremelimumab.

Information regarding tremelimumab (or antigen-binding fragments thereof) for use in the methods provided herein can be found in U.S. Pat. No. 6,682,736 (in which tremelimumab is referred to as 11.2.1), the disclosure of which is incorporated herein by reference in its entirety. Tremelimumab (also known as CP-675,206, CP-675, CP-675206, and ticilimumab) is a human IgG2 monoclonal antibody that is highly selective for CTLA4 and blocks binding of CTLA4 to CD80 (B7.1) and CD86 (B7.2). It has been shown to result in immune activation in vitro and some patients treated with tremelimumab have shown tumor regression.

Tremelimumab for use in the methods provided herein comprises a heavy chain and a light chain, or a heavy chain variable region and a light chain variable region. In a specific aspect, tremelimumab or an antigen-binding fragment thereof for use in the methods provided herein comprises a light chain variable region comprising the amino acid sequence of SEQ ID NO: 11 and a heavy chain variable region comprising the amino acid sequence of SEQ ID NO: 12. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 13-15, and wherein the light chain variable region comprises the Kabat-defined CDR1, CDR2, and CDR3 sequences of SEQ ID NOs: 16-18. Those of ordinary skill in the art would readily be able to identify Chothia-defined, Abm-defined, Kabat-defined, or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

In certain aspects, a subject presenting with a bladder cancer, namely, a UC, is administered durvalumab or an antigen-binding fragment thereof and tremelimumab or an antigen-binding fragment thereof. Durvalumab or an antigen-binding fragment thereof and tremelimumab or an antigen-binding fragment thereof can be administered only once or infrequently while still providing benefit to the patient. In further aspects, additional, follow-on doses are administered to the subject. Follow-on doses can be administered at various time intervals depending on the subject's age, weight, clinical assessment, tumor burden, and/or other factors, including the judgment of the attending physician, clinician, or medical practitioner.

The intervals between doses of durvalumab or an antigen-binding fragment thereof can be every two weeks (Q2W), every three weeks (Q3W), or every four weeks (Q4W). The intervals between doses of tremelimumab or an antigen-binding fragment thereof can be every four weeks. The intervals between doses of tremelimumab or an antigen-binding fragment thereof can be every two weeks, every three weeks, every four weeks, etc., up to every twelve weeks, for up to four doses per cycle. In a certain embodiment, durvalumab or an antigen-binding fragment thereof is administered to a subject having bladder cancer, e.g., UC, at a dose of 1500 mg (1.5 g) every 4 weeks (1500 mg Q4W, IV) in combination with tremelimumbab administered at a dose of 75 mg every four weeks (75 mg Q4W, IV) for up to four doses/cycle each. In an embodiment, this combination treatment is followed by administration of durvalumab at a dose of 1500 mg (1.5 g) every four weeks (1500 mg Q4W, IV). In an embodiment, the subject with bladder cancer, e.g., UC, is identified as having cancer or tumor cells or tissue that are PD-L1low/neg; PD-L1low; or PD-L1high for PD-L1 expression.

In some embodiments, at least two doses of durvalumab or an antigen-binding fragment thereof and tremelimumab or an antigen-binding fragment thereof are administered to the subject. In some embodiments, at least three doses, at least four doses, at least five doses, at least six doses, at least seven doses, at least eight doses, at least nine doses, at least ten doses, or at least fifteen doses or more of one or both of the anti-PD-L1 and/or the anti-CTLA4 antibodies can be administered to the subject. In some embodiments, durvalumab or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, tremelimumab or an antigen-binding fragment thereof is administered over a four-week treatment period, over an eight-week treatment period, over a twelve-week treatment period, over a sixteen-week treatment period, over a twenty-week treatment period, over a twenty-four-week treatment period, over a thirty-six-week treatment period, over a forty-eight-week treatment period, or over a one-year or more treatment period. In a particular embodiment, durvalumab is administered Q2W or Q4W until the subject responds, until disease progression, or until unacceptable toxicity is encountered. In another particular embodiment, durvalumab is co-administered with tremelimumab Q2W or Q4W until the subject responds, until disease progression, or until unacceptable toxicity is encountered.

The amount of durvalumab or an antigen-binding fragment thereof and the amount of tremelimumab or an antigen-binding fragment thereof to be administered to the subject with bladder cancer, in particular, UC, will depend on various parameters such as the patient's age, weight, clinical assessment, tumor burden and/or other factors, including the judgment of the attending physician.

In a certain aspect, a subject with bladder cancer, in particular, UC, is administered a 1 mg/kg to 20 mg/kg dose of durvalumab or an antigen-binding fragment thereof, in particular, a 1.5 g dose, every 2 weeks (Q2W) in combination with tremelimumab or an antigen-binding fragment thereof at a dose of 1 mg/kg to 5 mg/kg, in particular, a 75 mg dose, Q4W for up to 4 doses/cycle. In a certain aspect, a subject with bladder cancer, in particular, UC, is administered a 1 mg/kg to 20 mg/kg dose of durvalumab or an antigen-binding fragment thereof, in particular, a 1.5 g dose, every 4 weeks (Q4W) in combination with tremelimumab or an antigen-binding fragment thereof at a dose of 1 mg/kg to 5 mg/kg, in particular, a 75 mg dose, Q4W for up to 4 doses/cycle. In each of these aspects, the patient is administered a 1.5 g dose of durvalumab or an antigen-binding fragment thereof Q4W following the administration of tremelimumab. In an embodiment, the antibodies are administered to the subject by intravenous infusion.

The anti-PD-L1 antibody or an antigen binding fragment thereof, such as durvalumab, and/or the anti-CTLA4 antibody or an antigen binding fragment thereof, such as tremelimumab, may be administered in the described methods by any acceptable route of administration known and used in the relevant art. Without limitation, administration of one or more of the antibodies may be via an intravenous (e.g., intravenous infusion), parenteral, or subcutaneous route of administration. In a particular embodiment, the administration is by intravenous (IV) infusion.

In certain aspects, 10 mg/kg of durvalumab or an antigen-binding fragment thereof is administered to a subject having bladder cancer, such as UC, every two weeks (Q2W), for example, by intravenous infusion, until the subject responds to the anti-PD-L1 antibody treatment (e.g., achieves ORR, OS, PFS, or CR), until disease progression, or until unacceptable toxicity. In an embodiment, the intravenous infusion occurs over 60 minutes. In an embodiment, the durvalumab is administered in combination with an anti-CTLA1 antibody, such as tremelimumab, either at the same time or at different, predetermined times or administration cycles.

In certain aspects, 1.5 g of durvalumab or an antigen-binding fragment thereof is administered to a subject having bladder cancer, such as UC, every four weeks (Q4W), for example, by intravenous infusion, until the subject responds to the anti-PD-L1 antibody treatment (e.g., achieves an ORR, OS, PFS, or CR), until disease progression, or until unacceptable toxicity. In an embodiment, the durvalumab is administered in combination with an anti-CTLA1 antibody, such as tremelimumab, either at the same time or at different, predetermined times or administration cycles.

In certain aspects, 1.5 g of durvalumab or an antigen-binding fragment thereof is administered to a subject having bladder cancer, such as UC, every four weeks (Q4W), in combination with the administration of 75 mg of tremelimumab or an antigen binding fragment thereof, every four weeks (Q4W), for up to 4 doses per administration cycle. The administration of 1.5 g of durvalumab Q4W is followed by the administration of 75 mg of tremelimumab Q4W. In an embodiment, administration is by intravenous infusion. In certain aspects, the combination of durvalumab or an antigen-binding fragment thereof and tremelimumab or an antigen binding fragment thereof is administered until the subject responds to the treatment (e.g., achieves ORR, OS, PFS, or CR), until disease progression, or until unacceptable toxicity.

The methods provided herein are shown to decrease or retard bladder cancer tumor growth, and in particular, urothelial carcinoma tumor growth. In some aspects the tumor growth reduction or retardation can be statistically significant. A reduction in bladder cancer (e.g., UC) tumor growth can be measured by comparison of the growth of a subject's tumor at a baseline time, to an expected tumor growth, to an expected tumor growth based on a large patient population, or based on the tumor growth of a control population. In addition, a reduction in bladder cancer (e.g., UC) tumor growth (or size) can be measured by comparing the growth or size of the subject's tumor at a given time following treatment of the subject with the anti-PD-L1antibody (e.g., durvalumab) or an antigen binding fragment thereof, alone or in combination with the anti-CTLA4 antibody (e.g., tremelimumab) or an antigen binding fragment thereof, compared with the growth or size of the tumor at a baseline time.

In certain aspects, a tumor response is measured using the Immune-related Response Criteria (irRc). In certain aspects, a tumor response is measured using the Response Evaluation Critera in Solid Tumors (RECIST). These response measurement techniques are known and practiced by those having skill in the art.

In certain aspects, a subject's response to the bladder cancer treatment is detectable at about 1.5 to 7.5 months. In certain aspects, a subject's response to the bladder cancer treatment is detectable by about week 4, week 5, week 6, week 7, week 8, week 9, week 10, week 11, week 12, week 13, week 14, week 15, week 16, week 17, week 18, week 19, or week 20 or therebetween. In certain aspects, a subject's response to the bladder cancer treatment is detectable at week 25, week 28, week 30, week 33, week 35, week 40, week 45, or week 50 or therebetween. In certain aspects, a subject's response to the bladder cancer treatment is detectable at week 60, week 65, week 70, or beyond. In certain aspects, a subject's response to the bladder cancer treatment is durable such that subjects remain in response for 6 months, 7 months, 8 months, 9 months, 10 months, 12 months and longer during treatment according to the present methods.

In certain aspects, a subject treated according to the described methods achieves disease control (DC), which also serves to refer to the subject's response. Disease control can be a complete response (CR), partial response (PR), or stable disease (SD). A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR. A “partial response” (PR) refers to a decrease in tumor burden ≥30% relative to baseline. Confirmation can be obtained using a consecutive repeat assessment at least 4 weeks from the date of first documentation. “Stable disease” (SD) indicates a decrease in tumor burden of less than about 30% relative to baseline cannot be established and a 20% or greater increase compared to nadir cannot be established.

In certain aspects, administration of durvalumab or an antigen-binding fragment thereof, either alone as a monotherapy, or in combination with tremelimumab or an antigen-binding fragment thereof, or in combination with a standard of care therapy regimen, can increase progression-free survival (PFS) of the subject undergoing treatment. In certain aspects, administration of durvalumab or an antigen-binding fragment thereof, either alone as a monotherapy, or in combination with tremelimumab or an antigen-binding fragment thereof, or in combination with a standard of care therapy regimen, can increase the overall survival (OS) of the subject undergoing treatment.

In some embodiments, the subject has previously received treatment with at least one chemotherapeutic agent. In some embodiments, the subject has previously received treatment with at least two chemotherapeutic agents. The chemotherapeutic agent can be, for example, and without limitation, Vemurafenib, Erlotinib, Afatinib, Cetuximab, Carboplatin, Bevacizumab, Erlotinib, Gefitinib, and/or Pemetrexed. In certain embodiments, the subject has received a standard of care (SoC) treatment comprising, for example and without limitation, cisplatin, carboplatin, cisplatin+gemcitabine, or carboplatin+gemcitabine doublet combination. (See, e.g., R. Nagourney et al., 2008, Clin. Breast Cancer, Vol. 8, No. 5:432-435).

In some embodiments, the subject has an Eastern Cooperative Oncology Group (ECOG) (Oken M M, et al. Am. J. Clin. Oncol. 5: 649-55 (1982)) performance status of 0, 1, or 2 prior to the administration of durvalumab or an antigen-binding fragment thereof, or of a combination of durvalumab or an antigen-binding fragment thereof and tremelimumab or an antigen-binding fragment thereof.

As provided herein, durvalumab or an antigen-binding fragment thereof can also decrease free (soluble) PD-L1 levels. Free (soluble) PD-L1 refers to PD-L1 that is not bound (e.g., by durvalumab). In some embodiments, sPD-L1 levels are reduced and/or undetectable following administration of durvalumab or an antigen-binding fragment thereof, or following the administration of a combination of durvalumab or an antigen-binding fragment thereof and tremelimumab or an antigen-binding fragment thereof. In some embodiments, administration of durvalumab or an antigen-binding fragment thereof, or administration of a combination of durvalumab or an antigen-binding fragment thereof and tremelimumab or an antigen-binding fragment thereof reduces the rate of increase of free (soluble) PD-L1 levels as compared, for example, to the rate of increase of free (soluble) PD-L1 levels prior to the described administrations.

Treatment of a subject with a bladder cancer or tumor, such as UC, using durvalumab or an antigen-binding fragment thereof, or a combination of durvalumab or an antigen-binding fragment thereof and tremelimumab or an antigen-binding fragment thereof (i.e., co-therapy) as provided herein may in some cases result in an additive and/or a synergistic effect. As used herein, the term “synergistic” refers to a combination of therapies (e.g., a combination of durvalumab or an antigen-binding fragment thereof and tremelimumab or an antigen-binding fragment thereof; or a combination of durvalumab or an antigen-binding fragment thereof and another anti-cancer therapy, or SoC therapy), which is more effective than the additive effects of the single therapies alone.

A synergistic effect of a combination of therapies (e.g., a combination of durvalumab or an antigen-binding fragment thereof and tremelimumab or an antigen-binding fragment thereof) permits the use of lower dosages of one or more of the therapeutic agents and/or less frequent administration of the therapeutic agents to a subject with a bladder cancer or tumor, such as UC. The ability to utilize lower dosages of therapeutic agents and/or to administer such therapeutic agents less frequently reduces the toxicity associated with the administration of the therapies to a subject without reducing the efficacy of the therapies in the treatment of a bladder cancer or tumor, such as UC. In addition, a synergistic effect can result in improved efficacy of therapeutic agents in the management, treatment, or amelioration of a solid bladder cancer tumor. The synergistic effect of a combination of therapeutic agents can avoid or reduce adverse or unwanted side effects associated with the use of each therapy used singly (as monotherapy), e.g., at a higher dose.

In co-therapy, durvalumab or an antigen-binding fragment thereof may be optionally included in the same pharmaceutical composition as the tremelimumab or an antigen-binding fragment thereof, or the durvalumab or an antigen-binding fragment thereof may be included in a separate pharmaceutical composition. In this latter case, the pharmaceutical composition comprising durvalumab or an antigen-binding fragment thereof is suitable for administration prior to, simultaneously with, or following administration of the pharmaceutical composition comprising tremelimumab or an antigen-binding fragment thereof. In certain instances, the administration of durvalumab or an antigen-binding fragment thereof to a subject in one composition overlaps with the time of administration of tremelimumab or an antigen-binding fragment thereof in a separate composition.

The practice of the present invention employs, unless otherwise indicated, conventional techniques of molecular biology (including recombinant techniques), microbiology, cell biology, biochemistry, immunohistochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for certain embodiments will be discussed in the sections that follow.

The following examples are set forth to provide those of ordinary skill in the art with a complete disclosure and description of how to make and use the assay, screening, and therapeutic methods of the invention, and are not intended to limit the scope of what the inventors regard as their invention.

### EXAMPLE 1

### Properties of Durvalumab Based on Clinical Analyses

This Example provides an overview of the clinical pharmacology of durvalumab and demonstrates that this anti-PD-L1 antibody or an antigen binding fragment thereof exhibits properties and characteristics that support its suitability for use in the described methods of treating bladder cancer, such as urothelial carcinoma (UC), in a subject having bladder cancer, such as UC, and in need thereof.

### A. Pharmacokinetics (PK) of Durvalumab

The pharmacokinetics (PK) of durvalumab was studied in 1,337 patients with solid tumors who received durvalumab at doses ranging from 0.1 to 10 mg/kg administered Q2W, or 15 mg/kg administered every 3 weeks (Q3W), or 20 mg/kg administered every 4 weeks (Q4W). Following durvalumab treatment at 10 mg/kg Q2W, PK was expected to be in the linear range and in line with typical monoclonal antibodies (mAbs). Key results of the PK analyses demonstrated the following:

Durvalumab exhibited nonlinear PK at doses <3 mg/kg, likely due to saturable target-mediated clearance and exhibited linear PK at doses ≥3 mg/kg. The area under the concentration-time curve (AUC; 0 to 14 days) increased more than dose-proportionally in the dose range of 0.1 to 3 mg/kg and increased dose-proportionally at doses ≥3 mg/kg. Maximum concentration (Cₘₐₓ) increased in a dose-proportional manner within the dose range examined.

The steady state was achieved after around Week 16 of repeated dosing. Systemic accumulation in Cₘₐₓ and trough concentration (C_{trough}) was 0.64- to 1.87-fold and 3.39-to 4.93-fold, respectively, over the dose range examined.

The mean systemic linear clearance and central volume of distribution were 226 mL/day and 3.51 L with a modest between-subject variability of 29.3% and 21.2%, respectively. The estimated half-life was about 21 days following durvalumab administered at a dose of 10 mg/kg Q2W.

The durvalumab concentration describing half-maximal capacity for nonlinear clearance (Km) was approximately 0.5 μg/mL. Based on this estimate, >99% target saturation was expected at approximately 50 μg/mL concentration of durvalumab. The dose of 10 mg/kg was expected to achieve this target exposure in >95% of patients.

No dose adjustment was recommended in the proposed dose of durvalumab 10 mg/kg Q2W IV based on patient baseline demographics or disease characteristics. Based on population PK analysis, there was no clinically significant effect of age, body weight, gender, positive anti-drug antibodies (ADA), status, albumin levels, LDH levels, creatinine levels, soluble PIMA (sPD-L1) levels, tumor type, race, mild to moderate renal impairment, mild hepatic impairment, or ECOG status on the PK of durvalumab.

Population PK modeling supported the use of a potential fixed dosing regimen of 750 mg Q2W IV or an equivalent but less frequent fixed dosing regimen of 1500 mg Q4W IV.

### B. Immunogenicity of durvalumab

Overall, durvalumab demonstrated a low incidence (2.6%; 37/1,412) of treatment-emergent ADAs, with no clinically significant effect on PK following a 10 mg/kg Q2W IV dose.

In Study 1108 (ClinicalTrials.gov Identifier: NCT01693562), described in Example 2 below, 25 (2.5%) of 1,012 patients who were treated with durvalumab at doses ranging from 0.1 to 20 mg/kg tested positive for treatment-emergent ADAs, and three (0.3%) patients had neutralizing antibodies. Of 970 patients who were treated with durvalumab at 10 mg/kg Q2W, including 191 patients in the UC cohort, 21 (2.2%) patients tested positive for treatment-emergent ADA and neutralizing antibodies were detected in only 1 (0.1%) patient. The ADA titers ranged between 1 and 32 (75% range, 1 to 4 [n=43], 21% range, 8 to 32 [n=12]) for most positive ADA samples. The presence of ADA did not appear to have a clinically significant effect on PK.

By comparison, in a study involving the treatment of patients having non-small cell lung cancer (NSCLC), 16 (3.6%) of 442 patients who were treated with durvalumab at 10 mg/kg Q2W tested positive for treatment-emergent ADAs, and 2 (0.45%) patients had neutralizing antibodies. In that study, ADA titers were low and ranged from 1 to 16 for 95% (18/19) of samples (1 sample had a titer of 1,024). The presence of ADA did not appear to have a clinically significant effect on PK, safety, or efficacy in that study.

### C. Pharmacodynamics and PK-PD Relationship

Target engagement: A dose-dependent suppression of free soluble PD-L1 (sPD-L1) was observed following IV administration of durvalumab. Following administration of durvalumab, complete sPD-L1 suppression was observed around dose levels 0.3 mg/kg. Following durvalumab administration at 10 mg/kg Q2W, >93% of patients demonstrated complete sPD-L1 suppression throughout the dosing interval.

Pharmacodynamic biomarkers: Average proliferating CD8+ T-cell quantities were significantly elevated above the mean of the range of variability on Day 10 (mean±standard of the mean [SEM]: 110%±27%) and Day 15 (mean ±SEM: 67%±15%) following administration of 10 mg/kg durvalumab. Similar trends were observed for proliferating CD4+ T cells on Day 10 (mean±SEM: 64%±18%), although the magnitude of the increase was not significantly elevated above the range of variability (RV). No other lymphocyte populations demonstrated mean changes from baseline that exceeded the RV during the first 100 days of the trial. These data demonstrate a pharmacodynamic effect that is consistent with the proposed mechanism of action of durvalumab.

Effect of exposure on efficacy: No association was identified between individual observed PK exposures (concentration at end of infusion of Dose 1 [C_{max,1}], pre-infusion at Dose 1 or 2 [C_{min,2}], pre-infusion at steady state [C_{min,ss}]) and clinical efficacy endpoints (ORR and DoR assessed by BICR) for patients treated with durvalumab at 10 mg/kg Q2W IV in the UC cohort of the study described herein (Study 1108, Example 2). These findings are in agreement with the exposure-response analysis.

Effect of exposure on safety: No association between individual observed PK exposure (C_{max,1}, C_{min,2}, and C_{min,ss}) and occurrence of adverse events ((AEs), treatment-related AE, AESI, treatment-related AESI (with further breakdown by any grade, ≥Grade 2, ≥Grade 3), and AEs leading to permanent discontinuation of durvalumab) was identified in patients treated with durvalumab at 10 mg/kg Q2W IV in Study 1108. A few inverse (lower AEs with higher exposure) and increasing (higher AEs with higher exposure) trends were observed. These trends appeared to be random variations and were not considered to be clinically relevant since trends were often contradictory and were not consistent across trials, type of AEs, or PK metric.

Dose rationale: The proposed dosing regimen of durvalumab at 10 mg/kg Q2W was supported by a robust dataset obtained from clinical and nonclinical trials. Some of the key features included the following: (i) Durvalumab treatment was well tolerated. No dose-limiting toxicities were observed at durvalumab doses of 10 mg/kg Q2W or 20 mg/kg Q4W; (ii) Clinically meaningful efficacy, as discussed in Example 2 in connection with the UC cohort of the clinical study (Study 1108); (iii) Achievement of target exposure based on clinical PK/PD: durvalumab PK approached linearity at ≥3 mg/kg Q2W dose (trough concentration approximately 50 μg/mL), indicating complete target saturation. Population PK simulations indicated that following a dose regimen of durvalumab at 10 mg/kg Q2W, >95% of the patients would achieve the target trough concentration of ≥50 μg/mL to achieve complete target saturation; (iv) Achievement of target exposure based on nonclinical efficacy models: durvalumab at 10 mg/kg Q2W achieved a median trough concentration of >100 μg/mL (identified as a target concentration of durvalumab that yielded maximum tumor growth inhibition in mouse models); (v) No apparent association of PK exposure with efficacy or safety: no exposure-efficacy relationship was identified in the UC cohort of Study 1108 and no clear exposure-safety relationship was identified in the Study 1108 (or in a study involving anti-PD-L1antibody treatment of NSCLC) safety populations following the administration of durvalumab at 10 mg/kg; and (vi) a 2.6% (37/1,412 patients) ADA incidence was observed following administration of durvalumab at 10 mg/kg Q2W, with no clinically significant effect on PK. Thus, observed clinical activity in UC patients coupled with a manageable safety profile and supportive translational correlates supported the administration of durvalumab at a dose of 10 mg/kg Q2W IV for treatment of subjects having UC.

### D. Duration of Treatment

While an optimal duration for anti-PD-1/PD-L1 therapy is still unclear, the duration of anti-PD-1/PD-L1 treatment in clinical trials varies from up to 12 months, 24 months, or until disease progression. Without wishing to be bound by theory, because anti-PD-1/PD-L1 antibodies primarily biologically block the interaction of PD-L1 with PD-1 without altering the expression of PD-L1 on tumor cells (TC) or on tumor-associated immune cells (IC), continuous blockade may optimally prevent T-cell exhaustion. Previous protocols involving durvalumab used a limited duration of treatment with the option for re-treatment. As an acceptable safety profile was demonstrated for long-term dosing and the occurrence of new AEs appeared to plateau before 12 months of treatment, all durvalumab protocols embrace continuous treatment until disease progression. On balance, the potential benefit of allowing patients who are benefiting from durvalumab to continue treatment beyond 12 months is justified based on the ongoing risks. For this reason, the study described in Example 2 includes treatment of a subject's bladder cancer (e.g., UC) until disease progression.

### EXAMPLE 2

### Clinical Trial Involving the Treatment of Subjects Having Bladder Cancer (UC) with Durvalumab

### A. Overall Trial Design

The data related to the methods described herein are provided by ongoing Study 1108, a Phase 1/2, single-arm, open-label, multicenter clinical trial (ClinicalTrials.gov Identifier: NCT01693562). The dose-expansion phase of the Study included 17 different tumor-specific cohorts, including a urothelial bladder cancer (UC) cohort. Patients in the dose-expansion phase were treated and continue to be treated with durvalumab at 10 mg/kg Q2W.

A UC expansion cohort with no limitation on prior lines of therapy was initiated in which approximately 60 patients having histologically or cytologically confirmed UC were to be enrolled; the first 20 patients were enrolled regardless of PD-L1 expression, and the subsequent 40 patients were required to have ≥5% of their tumor cells positive for PD-L1 expression. Thereafter, 132 additional patients having histologically or cytologically confirmed inoperable or metastatic transitional cell (including transitional cell and mixed transitional cell/non-transitional cell histologies) carcinoma of the urothelium (including the urinary bladder, ureter, urethra, and renal pelvis) were to be enrolled regardless of PD-L1 expression; these patients received 1 to 2 prior systemic therapies, including a platinum-based regimen. In particular, these patients must have received and have progressed or were refractory to at least 1, but not more than 2, prior lines of systemic therapy for inoperable or metastatic disease, including a standard platinum-based regimen. Prior definitive chemoradiation for locally advanced disease, adjuvant treatment, or neoadjuvant treatment were considered to be a prior line of therapy, provided that progression had occurred <12 months from therapy (for chemoradiation and adjuvant treatment) or <12 months from surgery (for neoadjuvant treatment). Interval progression between 2 lines of therapy defines separate lines of therapy.

The primary endpoint for the UC cohort was confirmed objective response (OR) according to RECIST v1.1 as determined by blinded independent central review (BICR). The key secondary endpoints included DoR, disease control rate (DCR), PFS, and OS.

### B. Patient Population

Eligible patients were ≥18 years of age with histologically or cytologically confirmed locally advanced or metastatic UC. The eligibility criteria based on prior lines of therapy are as set forth in (A) above. Patients had an ECOG performance status score of 0 or 1, adequate organ and hematologic functions, and fresh tumor biopsy and/or archival tumor tissue available for PD-L1testing. The trial excluded patients who had a history of immunodeficiency; medical conditions that required systemic immunosuppression; history of severe immune-mediated adverse reactions; untreated central nervous system metastases; and human immunodeficiency virus, active tuberculosis, or hepatitis B or C infection.

### C. Efficacy Endpoints and Analyses

### Interim Efficacy Analysis

An interim analysis of efficacy was conducted after 103 UC patients were treated with durvalumab 10 mg/kg Q2W and had an opportunity to be followed for at least 13 weeks (i.e., had an opportunity to receive at least 2 follow-up scans assessed according to RECIST v 1.1, at Weeks 6 and 12, plus a 1-week visit window).

The analysis populations used for the interim efficacy analyses were a Primary Efficacy Population, defined as treated UC patients who had an opportunity to be followed for ≥13 weeks by the DCO date (i.e., had received the first dose of durvalumab ≥13 weeks prior to the DCO date). This was the primary analysis population for the interim+analysis of all efficacy data; and a Supportive Efficacy Population, defined as all treated UC patients who had an opportunity to be followed for ≥24 weeks by the DCO date (i.e., had received the first dose of durvalumab ≥24 weeks prior to the DCO date). This was the supportive analysis population for the interim efficacy analysis.

### Primary Efficacy Analysis

The primary efficacy endpoint was objective response (OR), defined as a best overall response of confirmed CR or partial response (PR) according to RECIST v1.1 as determined by BICR. ORR, defined as the proportion of patients with OR, was calculated, and the 95% exact two-sided CIs of ORR was estimated using the Clopper-Pearson method. Patients were scanned for disease assessment at baseline; Weeks 6, 12, and 16 following initiation of durvalumab therapy; and then every 8 weeks during treatment. Following discontinuation of treatment, patients were scanned every 2 months for 1 year, and then every 3 months until confirmed objective disease progression.

The primary interim analysis of ORR (exact 95% CI) was performed for all treated UC patients who had an opportunity to be followed for at least 13 weeks (the above-described Primary Efficacy Population). A supportive analysis of ORR was performed for all treated UC patients who had an opportunity to be followed for at least 24 weeks (Supportive Efficacy Population as described above). In addition, similar analyses of ORR were performed for a subpopulation of all treated UC patients who had progressed while on or after a platinum-based therapy, including those patients who had progressed within 12 months of receiving therapy in a neoadjuvant/adjuvant setting (2L post platinum).

### Secondary Efficacy Analyses

Secondary efficacy endpoints included DoR, DCR, time to response, PFS, change in target lesion size according to RECIST v1.1 as determined by BICR and investigator assessment, and OS. To assess PD-L1 expression in relation to the antitumor activity of durvalumab in UC patients, efficacy data were also analyzed by identified subgroups based on PD-L1 expression as determined evaluation of membranous PD-L1 staining in tumor cells (TC) and overall PD-L1staining of tumor-associated immune cells (ICs) by pathologists trained in assessment of the validated VENTANA PD-L1 (SP263) assay. A total of 103 UC patients provided a width between the observed ORR and its lower limit of the exact two-sided 95% CI, ranging from 7% to 9%, when the ORR was expected to be in the 20% to 30% range.

### D. Assessment of PD-L1 Expression Using a Validated PD-L1 Detection Assay

A scoring algorithm for differentiating responding and non-responding patients based upon a PD-L1 expression cutoff was developed by the inventors. The relationship between PD-L1expression levels on both tumor cells (TC) and immune cells (IC), and response to durvalumab therapy was evaluated in tumor samples from treated patients in the UC cohort of Study 1108. This analysis established the optimal algorithm to be a combined assessment of PD-L1staining of TC and IC.

To determine the primary PD-L1 subgroups for analysis, a cutoff was established for responders to durvalumab monotherapy. This cutoff was determined using a training data set consisting of the initial group of subjects enrolled, which were followed for a minimum of 12 weeks. PD-L1 expression data and ORR data based on RECIST v1.1 assessed by investigators from these subjects were used to define the PD-L1 expression threshold that best differentiated responding and non-responding subjects. Other RECIST v1.1 data by investigators (such as percent change from baseline in target lesions) and clinical data (such as discontinuation due to PD or early death) were taken into consideration. The optimal algorithm classified patients as having either PD-L1 high tumors when baseline PD-L1 expression was ≥25% either on TC or IC; or PD-L1 low/negative tumors when baseline PD-L1 expression was <25% on TC and IC. Based on this cutoff, a first version of the algorithm was defined (Development Algorithm).

The rationale for a 25% cutoff for TC was based on:

1. Biology of PD-L1 and response: high membranous tumor cell PD-L1 expression has been shown to be correlated with better clinical response with drugs targeting the PD-1/PD-L1 pathway

2. Prevalence in UC: evaluation of PD-L1 expression in commercial bladder cancer samples, where PD-L1 was found to be highly expressed in TC of some cases, suggesting an innate or adaptive overexpression of PD-L1; and,

3. Clinical outcome data in UC: one of the subjects with partial response (PR) and another subject with a best change of −38.5 had PD-L1≥25% in TC. Similarly, the rationale for 25% cutoff for IC was based on:

1. Biology of PD-L1 and response in UC: high PD-L1 expression in IC has been shown to correlated with better clinical response with an anti-PD-L1 drug, atezolizumab;

2. Prevalence in UC: among commercial bladder cancer samples, PD-L1 was found to be highly expressed in ICs of many cases, suggesting an adaptive overexpression of PD-L1; and,

3. Clinical outcome data in UC: finding that all 4 subjects with PR had PD-L1≥25% in IC.

Another important factor considered for the choice of the 25% cutoff for both TC and IC was the expected degree of inter-reader precision (reproducibility between pathologists), based on the experiences of the Pathologists from MedImmune and Ventana Medical Systems. The ORR assessed by investigator according to RECIST v1.1 in the subjects of the Training in the PD-L1 high subjects (≥25% in TC or IC) was 36.4% (4/11, 95% CI: 10.9, 69.2), while the ORR in the PD-L1 low/negative subjects (<25% in TC and IC) was 0%. The combined algorithm delivers a higher negative predictive value (NPV) and higher true positive rate (TPR) than scoring with TC or IC alone.

The assay protocol for PD-L1 staining was locked prior to patient testing and the algorithm was locked following the read-out of the 20-patient training set. Later in the trial, and implemented at the start of the addition of patients (132) in the study, the Final Algorithm (Table 1) was deployed, which consisted of a modification to the Development Algorithm to enhance the precision of scoring in cases when PD-L1 expression was ≤1% on IC, as follows:

TABLE 1
Description of PD-L1 status

| PD-L1 status | Description |
|---|---|
| PD-L1 high (referred to | Tumor cell ≥25% or immune cell ≥25% |
| as PD-L1 positive in CSP) | |
| PD-L1 low/negative (referred | Tumor cell <25% and immune cell <25% |
| to as PD-L1 negative in CSP) | |

CSP = clinical study protocol; PD-L1 = programmed cell death ligand-1.
Note:
When the immune cell area represented ≤1% of the total tumor area, PD-L1 expression status was defined as follows: PD-L1 high when baseline PD-L1 expression was TC ≥25% or IC = 100%, and PD-L1 low/negative when baseline was TC < 25% and IC < 100%.

This modification to the algorithm enhanced the precision of scoring in cases with a low degree of IC infiltration while maintaining 100% concordance between the Development and Final Algorithms.

Application of the scoring algorithm to determine PD-L1 status (high versus low/negative) was performed programmatically based upon the raw data captured during the pathologist's evaluation of percentage of PD-L1 TC and IC stained cells. The Final Algorithm was applied prospectively in samples from 43 patients in the Primary Efficacy Population, and was applied retrospectively to 63 patients in the Primary Efficacy Population. While not intending to be limiting, the PD-L1 detection assay (VENTANA PD-L1 (SP263) assay), may serve as a complementary diagnostic assay, as it can provide an assessment of PD-L1 expression and useful information on the likelihood of response to durvalumab across the UC population.

The PD-L1 expression cutoff and initial scoring algorithm were defined based on a Training Set of 20 UC subjects (the first 20 UC subjects enrolled in Study 1108), and PD-L1high was originally defined as baseline PD-L1 expression with TC≥25% or IC≥25%, and PD-L1low/negative was defined as baseline PD L1 expression with TC<25% and IC<25% (Development Algorithm). Later in the study, the Final Algorithm was deployed, which consisted of a modification of the Development Algorithm—when the IC area represented <1% of the total tumor area, PD-L1 expression status was defined as follows: PD-L1 high when baseline PD-L1 expression was TC≥25% and or IC=100%, and PD-L1 low/negative when baseline PD L1 expression was TC <25% and IC<100%. The addition of this rule enhanced the precision of scoring in cases with low IC content.

### E. Patient Populations Studied

Of the 103 UC patients in the Primary Efficacy Population, 94 were 2L+ post-platinum patients who had progressed while on or after a platinum-based therapy, including those patients who progressed within 12 months of receiving therapy in a neo-adjuvant/adjuvant setting. The other 9 patients were either treatment-naive or were designated as 1L patients who had received platinum-based therapy in the neo-adjuvant/adjuvant setting and had progressed more than 12 months after the last dose of therapy. Seven of the 9 treatment-naive/1L patients were deemed cisplatin-ineligible based on cisplatin eligibility criteria utilized in the study.

The UC patient population treated in Study 1108 represented a clearly defined population with a high unmet clinical need and provided an intended population to be treated in clinical practice. The inclusion criteria were designed to define accurately a study population relevant to current clinical practice in the intended target population and to exclude patients whose participation would have been inappropriate for safety reasons. The median age of patients was in the mid-sixties; most patients were male and most had an ECOG status of 1 at study entry (Table 2). The majority of patients had received 1 or 2 prior treatment lines at study entry. All but 3 patients had had prior platinum-based therapy that was either cisplatin-based (69.9%) or carboplatin-based (27.2%). Baseline disease characteristics indicated a study population with poor prognosis (Table 2). Approximately 95% of the Primary Efficacy Population had visceral disease, including nearly 50% with liver metastases; 36.9% had received at least 2 prior lines of systemic therapy and 14.6% had received 3 or more prior lines of therapy.

TABLE 2
Demographics and baseline disease characteristics by PD-L1 status - UC cohort Study 1108 (Primary
Efficacy Population: treated patients with opportunity for ≥13 weeks of follow-up)

| | All UC | | | 2L+ post-platinum UC | | |
|---|---|---|---|---|---|---|
| | | PD-L1 | PD-L1 | | PD-L1 | PD-L1 |
| | Total | High ^{a} | Low/Neg ^{a} | Total | High ^{a} | Low/Neg^{a} |
| Parameter | N = 103 | N = 61 | N = 39 | N = 94 | N = 58 | N = 33 |

Age (years)

| | | | | | | | | | | | | |
|---|---|---|---|---|---|---|---|---|---|---|---|---|
| Median | 66.0 | (34, 88) | 67.0 | (34, 88) | 65.0 | (48, 82) | 66.0 | (34, 88) | 67.5 | (34, 88) | 65.0 | (48, 82) |
| (Min, max) | | | | | | | | | | | | |

Sex, n (%)

| | | | | | | | | | | | | |
|---|---|---|---|---|---|---|---|---|---|---|---|---|
| Female | 32 | (31.1%) | 18 | (29.5%) | 13 | (33.3%) | 28 | (29.8%) | 18 | (31.0%) | 9 | (27.3%) |
| Male | 71 | (68.9%) | 43 | (70.5%) | 26 | (66.7%) | 66 | (70.2%) | 40 | (69.0%) | 24 | (72.7%) |

ECOG, n (%)

| | | | | | | | | | | | | |
|---|---|---|---|---|---|---|---|---|---|---|---|---|
| 0 | 30 | (29.1%) | 19 | (31.1%) | 8 | (20.5%) | 27 | (28.7%) | 18 | (31.0%) | 6 | (18.2%) |
| 1 | 73 | (70.9%) | 42 | (68.9%) | 31 | (79.5%) | 67 | (71.3%) | 40 | (69.0%) | 27 | (81.8%) |

Site of disease at baseline, n (%) ^{b}

| | | | | | | | | | | | | |
|---|---|---|---|---|---|---|---|---|---|---|---|---|
| Visceral | 98 | (95.1%) | 58 | (95.1%) | 39 | (100%) | 89 | (94.7%) | 55 | (94.8%) | 33 | (100%) |

TABLE 2
Demographics and baseline disease characteristics by PD-L1 status - UC cohort Study 1108 (Primary
Efficacy Population: treated patients with opportunity for ≥13 weeks of follow-up)

| | All UC | | | | | | 2L+ post-platinum UC | | | | | |
|---|---|---|---|---|---|---|---|---|---|---|---|---|
| | | | PD-L1 | | PD-L1 | | | | PD-L1 | | PD-L1 | |
| | Total | | High ^{a} | | Low/Neg ^{a} | | Total | | High ^{a} | | Low/Neg ^{a} | |
| Parameter | N= 103 | | N = 61 | | N = 39 | | N = 94 | | N = 58 | | N = 33 | |
| Liver | 50 | (48.5%) | 30 | (49.2%) | 20 | (51.3%) | 46 | (48.9%) | 28 | (48.3%) | 18 | (54.5%) |

| | | | | | | | | | | |
|---|---|---|---|---|---|---|---|---|---|---|
| Lymph | 5 | (4.9%) | 3 | (4.9%) | 0 | 5 | (5.3%) | 3 | (5.2%) | 0 |
| node only | | | | | | | | | | |

Prior line of therapy for inoperable/metastatic disease, n (%)

| 0 | 9 | (8.7%) | 3 | (4.9%) | 6 | (15.4%) | 0 | | 0 | | 0 | |
|---|---|---|---|---|---|---|---|---|---|---|---|---|
| 1 | 56 | (54.4%) | 35 | (57.4%) | 18 | (46.2%) | 56 | (59.6%) | 35 | (60.3%) | 18 | (54.5%) |
| 2 | 23 | (22.3%) | 15 | (24.6%) | 8 | (20.5%) | 23 | (24.5%) | 15 | (25.9%) | 8 | (24.2%) |
| 3 | 9 | (8.7%) | 4 | (6.6%) | 5 | (12.8%) | 9 | (9.6%) | 4 | (6.9%) | 5 | (15.2%) |
| ≥4 | 6 | (5.8%) | 4 | (6.6%) | 2(5.1%) | | 6 | (6.4%) | 4 | (6.9%) | 2 | (6.1%) |

Previous therapy with platinum-based regimen

| | | | | | | | | | | | | |
|---|---|---|---|---|---|---|---|---|---|---|---|---|
| Yes | 100 | (97.1%) | 61 | (100%) | 36 | (92.3%) | 94 | (100%) | 58 | (100%) | 33 | (100%) |
| Cisplatin- | 72 | (69.9%) | 46 | (75.4%) | 25 | (64.1%) | 68 | (72.3%) | 45 | (77.6%) | 22 | (66.7%) |
| based | | | | | | | | | | | | |
| Carboplatin- | 28 | (27.2%) | 15 | (24.6%) | 11 | (28.2%) | 26 | (27.7%) | 13 | (22.4%) | 11 | (33.3%) |
| based | | | | | | | | | | | | |

| | | | | | | |
|---|---|---|---|---|---|---|
| No | 3 | (2.9%) | 0 | 3 | (7.7%) | Not applicable |

2L+ = second line or greater; DCO = data cutoff; ECOG = Eastern Cooperative Oncology Group; IC = immune cell; max = maximum; min = minimum; PD-L1 = programmed cell death ligand-1; TC = tumor cell; UC = UC = urothelial carcinoma.
Patients received their first dose of durvalumab at least 13 weeks prior to the DCO date.
^{a} PD-L1 high was defined as TC ≥ 25% or IC ≥ 25%; PD-L1 low/neg was defined as TC < 25% and IC < 25%.
^{b} Site of disease at baseline was derived from the baseline disease assessment assessed by investigator and blinded independent central. Visceral metastasis included liver, lung, bone, or any non-lymph node or soft tissue metastasis.

### F. Efficacy

### Objective ResponseRate (ORR), Duration of Response (DoR), and Disease Control Rate By BICR

Durvalumab monotherapy demonstrated meaningful benefit in patients with locally advanced or metastatic UC, based on endpoints (ORR, DoR) that are likely to predict clinical benefit. In the Primary Efficacy Population, durvalumab monotherapy resulted in an ORR of 20.4% (95% CT: 13.1%, 29.5%) by BICR according to RECTST v1.1 in all UC patients and an ORR of 20.2% (95% CT: 12.6%, 29.8%) in the 2L+ post-platinum subgroup (Table 3). Three additional patients had an unconfirmed response per BICR at the time of DCO with potential of confirmation as follows: one of the three patients still on treatment had a PR that had been confirmed per BICR based on a subsequent scan after DCO; the second of the three patients still on treatment was assessed as a PR on 2 consecutive scans, but the interval between the 2 scans was 26 days (<4 weeks); and the third of the three patients still on treatment had an initial PR at the second scan (Week 12), with the potential of being confirmed.

The patient responses occurred early during treatment and were durable. Among the 21 responders, the median time to response was 1.41 month (range, 1.2 to 7.2). The median DoR was not reached (range, 1.4+ to 19.9 months). A total of 16 patients remained in response for at least 6 months; 7 patients remained in response for at least 9 months. Eighteen of the 21 responders (85.7%) had ongoing responses at the time of DCO. Of the 3 patients who progressed after an initial response, as assessed per BICR, all 3 completed 12 months of treatment with durvalumab dosed at 10 mg/kg Q2W, including 1 patient who progressed due to an increase in the target lesion on Day 286 (disease assessment 6). This patient had a subsequent CR per BICR starting on Day 392 (disease assessment 8) and maintained the ongoing CR through the last follow-up as of DCO on Day 650 (disease assessment 12). The 2L+ post-platinum UC subgroup showed similar efficacy (Table 3).

### G. PD-L1 subgroup analysis

Although durvalumab has demonstrated clinical activity and durability of response in both PD-L1 high and PD-L1 low/negative subgroups, the combined algorithm using TC/IC in the VENTANA PD-L1 (SP263) Assay did show a high TPR (85.7%) and a high NPV (92.3%). In contrast, the TC alone or IC alone algorithms are not as discriminating, as indicated by lower TPR and NPV. These data support the utility of the algorithm based on TC or IC combined to identify subjects who are more likely to respond to durvalumab. In addition, given the high NPV of the assay, it is especially helpful in informing patients on the likelihood of response to durvalumab, that is, subjects who are classified PD-L1 high with the VENTANA PD-L1 (SP263) Assay tended to have a higher ORR than subjects who were PD-L1 low/negative. The combined algorithm supports the utility of the assay to identify subjects most likely to respond to durvalumab but does not exclude completely subjects who may respond. The sponsor considered that the high NPV of the assay is especially helpful in informing patients on the likelihood of response to durvalumab but should not be utilized to exclude subjects from therapy, as three (3) PD-L1 low/negative durvalumab treated subjects had clinical responses (1 PR and 2 complete response), with responses still ongoing at the time of DCO.

Clinical activity was observed across the PD-L1 high and PD-L1 low/negative subgroups. The ORR was 29.5% (18/61; 95% CI: 18.5%, 42.6%) in the PD-L1 high subgroup and 7.7% (3/39; 95% CI: 1.6%, 20.9%) in the PD-L1 low/negative subgroup. CRs were observed across both subgroups: PD-L1 high, 3 (4.9%) patients, and PD-L1 low/negative, 2 (5.1%) patients. The responses observed in both subgroups were durable.

### H. Supportive Efficacy Analysis

In the Supportive Efficacy Population, the ORR was 28.6% (95% CI: 17.9%, 41.3%) for all UC patients and 29.6% (95% CI: 18.0%, 43.6%) for 2L+ post-platinum patients (Table 4). The median duration of response has not been reached. Similar to the Primary Efficacy Population, clinical activity was observed across the PD-L1 high and PD-L1 low/negative subgroups. The ORR was 40.0% (16/40; 95% Cl: 24.9%, 56.7%) in the PD-L1 high subgroup and 8.7% (2/23; 95% CT: 1.1%, 28.0%) in the PD-L1 low/negative subgroup. CRs were observed across both subgroups: PD-L1 high, 2 (5.0%) patients; and PD-L1 low/negative, 2 (8.7%) patients. The responses observed in both subgroups were durable.

TABLE 3
Efficacy per BICR according to RECIST v1.1 by PD-L1 status - UC cohort Study 1108 (Primary
Efficacy Population: treated patients with opportunity for ≥13 weeks of follow-up)

| | All UC | | | 2L+ post-platinum UC ^{c} | | |
|---|---|---|---|---|---|---|
| | | PD-L1 | PD-L1 | | PD-L1 | PD-L1 |
| | Total ^{a} | High ^{b} | Low/Neg ^{b} | Total | High ^{b} | Low/Neg ^{b} |
| Parameter | N = 103 | N = 61 | N = 39 | N = 94 | N = 58 | N = 33 |
| ORR, n (%) | 21 (20.4%) | 18 (29.5%) | 3 (7.7%) | 19 (20.2%) | 17 (29.3%) | 2(6.1%) |
| (95% CI) | (13.1%, 29.5%) | (18.5%, 42.6%) | (1.6%, 20.9%) | (12.6%, 29.8%) | (18.1%, 42.7%) | (0.7%, 20.2%) |

Best overall response

| CR | 5 | (4.9%) | 3 | (4.9%) | 2 | (5.1%) | 4 | (4.3%) | 3 | (5.2%) | 1 | (3.0%) |
|---|---|---|---|---|---|---|---|---|---|---|---|---|
| PR | 16 | (15.5%) | 15 | (24.6%) | 1 | (2.6%) | 15 | (16.0%) | 14 | (24.1%) | 1 | (3.0%) |
| DCR, n (%) | 44 (42.7%) | | 31 (50.8%) | | 10 (25.6%) | | 40 (42.6%) | | 29 (50.0%) | | 8 (24.2%) | |
| (95% CI) | (33.0%, 52.8%) | | (37.7%, 63.9%) | | (13.0%, 42.1%) | | (32.4%, 53.2%) | | (36.6%, 63.4%) | | (11.1%, 42.3%) | |
| Responses ongoing | 18 | (85.7%) | 15 | (83.3%) | 3 | (100.0%) | 16 | (84.2%) | 14 | (82.4%) | 2 | (100.0%) |
| at time of DCO | | | | | | | | | | | | |

Duration of response (months)

| Median | NR | | NR | | NR | | NR | | NR | | NR | |
|---|---|---|---|---|---|---|---|---|---|---|---|---|
| (95% CI) | (NE, NE) | | (6.2, NE) | | (NE, NE) | | (6.2, NE) | | (6.2, NE) | | (NE, NE) | |
| (min, max) | (1.4+, 19.9+) | | (1.5+, 19.9+) | | (1.4+, 9.6+) | | (1.4+, 19.9+) | | (1.5+, 19.9+) | | (1.4+, 9.6+) | |
| ≥6 months, n (%) | 16 | (76.2%) | 14 | (77.8%) | 2 | (66.7%) | 14 | (73.7%) | 13 | (76.5%) | 1 | (50.0%) |
| ≥9 months, n (%) | 7 | (33.3%) | 6 | (33.3%) | 1 | (33.3%) | 7 | (36.8%) | 6 | (35.3%) | 1 | (50.0%) |

| | | | | | | | | | | |
|---|---|---|---|---|---|---|---|---|---|---|
| ≥12 months, n (%) | 1 | (4.8%) | 1 | (5.6%) | 0 | 1 | (5.3%) | 1 | (5.9%) | 0 |

Time to response (months)

| | | | | | | |
|---|---|---|---|---|---|---|
| Median | 1.41 | 1.41 | 1.48 | 1.41 | 1.41 | 1.45 |
| (min, max) | (1.2, 7.2) | (1.2, 7.2) | (1.4, 2.7) | (1.2, 3.2) | (1.2, 3.2) | (1.4, 1.5) |

TABLE 3
Efficacy per BICR according to RECIST v1.1 by PD-L1 status -
UC cohort Study 1108 (Primary Efficacy Population: treated
patients with opportunity for ≥13 weeks of follow-up)

| | All UC | | | 2L+ post-platinum UC ^{c} | | |
|---|---|---|---|---|---|---|
| | | | PD-L1 | | | PD-L1 |
| | | PD-L1 | Low/ | | PD-L1 | Low/ |
| Parameter | Total ^{a} | High ^{b} | Neg ^{b} | Total | High ^{b} | Neg ^{b} |
| [symbol] | [symbol] | [symbol] | [symbol] | [symbol] | [symbol] | [symbol] |

2L+ = second line or greater; BICR = blinded independent central review; CI = confidence interval; CR = complete response; DCO = data cutoff; DCR = disease control rate including CR, PR or SD ≥ 6 weeks; IC = immune cell; min = minimum; max = maximum; NE = not estimable; neg = negative; NR = not reached; ORR = objective response rate; PD-L1 = programmed cell death ligand-1; PR = partial response; RECIST v1.1 = Response Evaluation Criteria in Solid Tumors version 1.1; SD = stable disease; TC = tumor cell; UC = urothelial carcinoma.
Patients received their first dose of durvalumab ≥13 weeks prior to the DCO date.
Response was determined by BICR based on RECIST v1.1.
^{a} The Total column includes 3 subjects who had unknown PD-L1 status and who are not included in either the PD-L1 high or PD-L1 low/neg subgroups. ^{b} PD-L1 high was defined as TC ≥ 25% or IC ≥ 25%; PD-L1 low/neg was defined as TC < 25% and IC < 25%.
^{c} 2L+ post-platinum patients had progressed while on or after a platinum-based therapy, including those patients who progressed within 12 months of receiving therapy in a neo-adjuvant/adjuvant setting. [symbol]
indicates data missing or illegible when filed

TABLE 4
Efficacy per BICR according to RECIST v1.1 by PD-L1 status - UC cohort Study 1108 (Supportive
Efficacy Population: treated patients with opportunity for ≥24 weeks of follow-up)

| | All UC | | | 2L+ post-platinum UC ^{b} | | |
|---|---|---|---|---|---|---|
| | | PD-L1 | PD-L1 | | PD-L1 | PD-L1 |
| | Total | High ^{a} | Low/Neg ^{a} | Total | High ^{a} | Low/Neg ^{a} |
| Parameter | N = 63 | N = 40 | N = 23 | N = 54 | N = 37 | N = 17 |
| ORR, n (%) | 18 (28.6%) | 16 (40.0%) | 2 (8.7%) | 16 (29.6%) | 15 (40.5%) | 1 (5.9%) |
| (95% CI) | (17.9%. 41.3%) | (24.9%. 56.7%) | (1.1%. 28.0%) | (18.0%, 43.6%) | (24.8%, 57.9%) | (0.1%, 28.7%) |

Best overall response

| | | | | | | |
|---|---|---|---|---|---|---|
| CR | 4 (6.3%) | 2 (5.0%) | 2 (8.7%) | 3 (5.6%) | 2 (5.4%) | 1 (5.9%) |
| PR | 14 (22.2%) | 14 (35.0%) | 0 | 13 (24.1%) | 13 (35.1%) | 0 |

TABLE 4
Efficacy per BICR according to RECIST v1.1 by PD-L1 status - UC cohort Study 1108 (Supportive
Efficacy Population: treated patients with opportunity for ≥24 weeks of follow-up)

| | All UC | | | | | | 2L+ post-platinum UC ^{b} | | | | |
|---|---|---|---|---|---|---|---|---|---|---|---|
| | | | PD-L1 | | PD-L1 | | | | PD-L1 | | PD-L1 |
| Parameter | Total | | High ^{a} | | Low/Neg ^{a} | | Total | | High ^{a} | | Low/Neg ^{a} |
| DCR, n (%) | 29 (46.0%) | | 23 (57.5%) | | 6 (26.1%) | | 25 (46.3%) | | 21 (56.8%) | | 4 (23.5%) |
| (95% CI) | (33.4%. 59.1%) | | (40.9%, 73.0%) | | (10.2%, 48.4%) | | (32.6%, 60.4%) | | (39.5%. 72.9%) | | (6.8%. 49.9%) |
| Responses ongoing | 15 | (83.3%) | 13 | (81.3%) | 2 | (100.0%) | 13 | (81.3%) | 12 | (80.0%) | 1 (100.0%) |
| at time of DCO | | | | | | | | | | | |

Duration of response (months)

| | | | | | | |
|---|---|---|---|---|---|---|
| Median | NR | NR | NR | NR | NR | NR |
| (95% CI) | (NE, NE) | (6.2, NE) | (NE, NE) | (6.2, NE) | (6.2. NE) | (NE. NE) |
| (min, max) | (4.2+, 19.9+) | (4.2+, 19.9+) | (6.6+, 9.6+) | (4.2+, 19.9+) | (4.2+, 19.9+) | (9.6+. 9.6+) |

Duration of response (%)

| | | | | | | | | | | | |
|---|---|---|---|---|---|---|---|---|---|---|---|
| ≥6 months | 16 | (88.9%) | 14 | (87.5%) | 2 | (100.0%) | 14 | (87.5%) | 13 | (86.7%) | 1 (100.0%) |
| ≥9 months | 7 | (38.9%) | 6 | (37.5%) | 1 | (50.0%) | 7 | (43.8%) | 6 | (40.0%) | 1 (100.0%) |

| | | | | | | | | | | |
|---|---|---|---|---|---|---|---|---|---|---|
| ≥12 months | 1 | (5.6%) | 1 | (6.3%) | 0 | 1 | (6.3%) | 1 | (6.7%) | 0 |

Time to response (months)

| | | | | | | |
|---|---|---|---|---|---|---|
| Median | 1.41 | 1.41 | 2.05 | 1.41 | 1.41 | 1.41 |
| (min, max) | (1.2. 7.2) | (1.2, 7.2) | (1.4, 2.7) | (1.2. 3.2) | (1.2, 3.2) | (1.4, 1.4) |

2L+ = second line or greater; BICR = blinded independent central review; CI = confidence interval; CR = complete response: DCO = data cutoff; DCR = disease control rate; IC = immune cell; max = maximum: min = minimum; NE = not estimable; neg = negative; NR = not reached; ORR = objective response rate; PD-L1 = programmed cell death ligand-1; PR = partial response; RECIST = Response Evaluation Criteria in Solid Tumors; SD = stable disease; TC = tumor cell; UC = urothelial carcinoma.
Patients received their first dose of durvalumab ≥ 24 weeks prior to the DCO date.
Response was determined by BICR based on RECIST v1.1.
^{a} PD-L1 high was defined as TC ≥ 25% or IC > 25%: PD-L1 low/neg was defined as TC < 25% and IC < 25%.
^{b} 2L+ post-platinum patients had progressed while on or after a platinum-based therapy, including those patients who progressed within 12 months of receiving therapy in a neo-adjuvant/adjuvant setting.

### I. Progression Free Survival by BICR

The median progression free survival (PFS) in the Primary Efficacy Population was 2.2 months (95% CI: 1.4, 2.7). The PFS rate at 6 months was 28.3% (95% CI: 19.3%, 37.9%). The median PFS was 2.5 months in the PD-L1 high subgroup and 1.5 months in the PD-L1low/negative subgroup. The PFS rate at 6 months was 39.2% (95% CI: 26.4%, 51.8%) for the PD-L1 high subgroup and 13.4% (95% CI: 4.2%, 28.1%) for the PD-L1 low/negative subgroup.

### J. Overall Survival

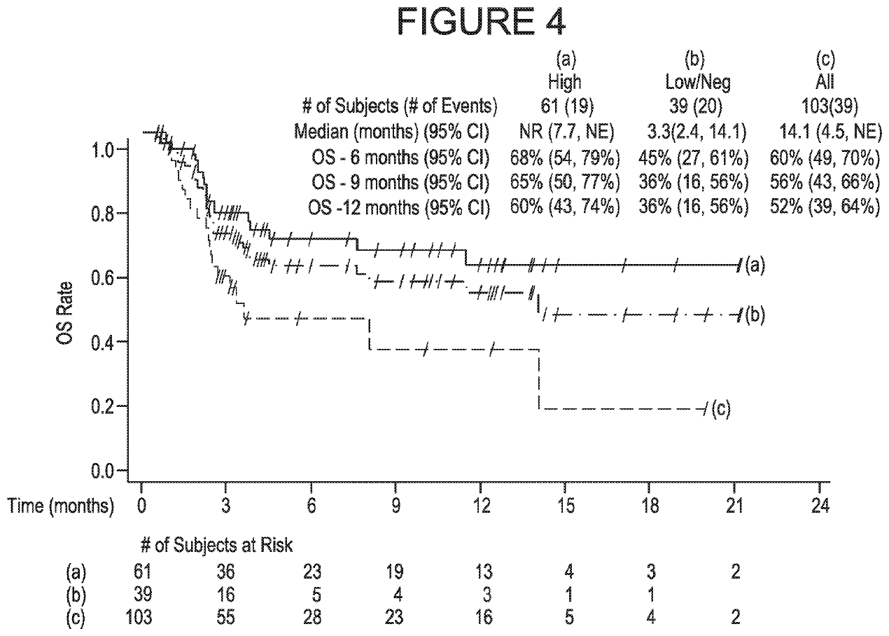

The median overall survival (OS) in the Primary Efficacy Population was 14.1 months (95% CI: 4.5, not estimable). The OS rate at 6 months, 9 months, and 12 months was 60.3% (95% CI: 48.7%, 70.1%), 55.7% (95% CI: 43.2%, 66.4%), and 52.4% (95% CI: 39.1%, 64.1%), respectively (Table 5). The median OS was not reached in the PD-L1 high group and was 3.3 months in the PD-L1 low/negative group. FIG. 4 shows a Kaplan-Meier estimate of OS.

TABLE 5
Overall survival (OS) by PD-L1 status - UC cohort Study
1108 (Primary Efficacy Population: treated patients
with an opportunity for ≥13 weeks of follow-up)

| | All UC | | |
|---|---|---|---|
| | | PD-L1 | PD-L1 |
| | Total ^{a} | High ^{b} | Low/Neg ^{b} |
| Parameter | N = 103 | N = 61 | N = 39 |
| Median | 14.1 | NR | 3.3 |
| OS (months) | (4.5, NE) | (7.7, NE) | (2.4, 14.1) |
| (95% CI) | | | |
| OS at 6 | 60.3% | 68.4% | 44.7% |
| months, % | (48.7%, 70.1%) | (53.6%, 79.3%) | (26.5%, 61.4%) |
| (95% CI) | | | |
| OS at 9 | 55.7% | 65.1% | 35.8% |
| months, % | (43.2%, 66.4%) | (49.6%, 76.9%) | (16.0%, 56.2%) |
| (95% CI) | | | |
| OS at 12 | 52.4% | 60.4% | 35.8% |
| months, % | (39.1%, 64.1%) | (43.3%, 73.9%) | (16.0%, 56.2%) |
| (95% CI) | | | |

CI = confidence interval; DCO = data cut-off; IC = immune cell; neg = negative; NE = not estimable; NR = not reached; OS = overall survival; PD-L1 = programmed cell death ligand-1; TC = tumor cell; UC = urothelial carcinoma.
^{a} The Total column includes 3 subjects who had unknown PD-L1 status and who are not included in either the PD-L1 high or PD-L1 low/neg subgroups.
^{b} PD-L1 high was defined as TC ≥ 25% or IC ≥ 25%; PD-L1 low/neg was defined as TC < 25% and IC < 25%.

### K. Efficacy by Subgroup

Objective responses (ORs) were observed across all subgroups based on baseline demographics or disease characteristics, including subsets with poor prognosis, such as patients with visceral metastasis or liver metastasis. In the Primary Efficacy Population, ORRs in subgroups for visceral metastasis, liver metastasis, and lymph node only were 19.4% (19/98; 95% CI: 12.1%, 28.6%), 10% (5/50; 95% CI: 3.3%, 21.8%) and 40% (2/5; 95% CI: 5.3%, 85.3%), respectively. While numerical differences in point estimates across some subgroups were noted, sample sizes were small and 95% CIs overlapped for most subgroup comparisons, except for tumor burden where patients with a tumor burden below the median had a higher ORR. Comparable ORRs were observed for key subgroups, including region, prior line of therapy, baseline creatinine clearance, site of primary tumor, and previous platinum therapy.

Data from another clinical study in which durvalumab was used in the treatment of NSCLC patients also supported a response and durability activity consistent with other anti-PD-1/PD-L1 therapies in patients with locally advanced or metastatic NSCLC who received at least 2 prior systemic treatment regimens. The NSCLC patient study adds supportive value to the clinical activity of durvalumab. For example, Cohort 2 of the NSCLC study included 265 patients with epidermal growth factor receptor/anaplastic lymphoma kinase wild-type NSCLC who were treated with durvalumab 10 mg/kg Q2W, and had mature data with a follow-up of >12 months. ORR was observed across PD-L1 high and PD-L1 low/negative subgroups with durable response, i.e., ORRs were 16.4% (95% CI: 10.8, 23.5) for the PD-L1 high subgroup and 7.5% (95% CI: 3.1, 14.9) for the PD-L1 low/negative subgroup. As of DCO, the median DoR was 12.3 months for patients in the PD-L1 high subgroup and was not reached in the PD-L1low/negative subgroup. In the NSCLC study, the median OS was 10.9 months and 9.3 months in PD-L1 high subgroup and PD-L1 low/negative subgroup, respectively.

### L. Conclusions of the Clinical Study

### Objective Response

Durvalumab monotherapy demonstrated meaningful benefit in patients with locally advanced or metastatic UC based on objective response rate (ORR) and duration of response (DoR), endpoints that can be predictive of clinical benefit relative to commonly used therapies. More specifically, in the Primary Efficacy Population (all UC patients who had an opportunity for ≥13 weeks of follow-up), the ORR was 20.4% (21/103; 95% CI: 13.1%, 29.5%) by BICR according to RECIST v1.1, including 5 patients (4.9%) with a best response of CR. The ORR in the 2L+ post-platinum UC patients was 20.2% (19/94; 95% CI: 12.6%, 29.8%). In addition, 3 patients (who were all still on treatment at the time of DCO) had an unconfirmed response at DCO. In the Supportive Efficacy Population (UC patients who had an opportunity for ≥24 weeks of follow-up), the ORR was 28.6% (18/63; 95% CI: 17.9%, 41.3%). The ORR in 2L+ post-platinum UC patients was 29.6% (16/54; 95% CI: 18.0%, 43.6%).

### Duration of Response

In Study 1108 for treating bladder cancer, responses occurred early in treatment and were durable. The median time to response was 1.41 months (range, 1.2 to 7.2 months). The median DoR was not yet reached (range, 1.4+ to 19.9+ months). As of the DCO, 18 (85.7%) of 21 responders had an ongoing response; 16 patients had a DoR >6 months; and 9 patients had a DoR >9 months. Of the 3 patients who progressed per BICR according to RECIST v1.1, all completed 12 months of treatment with durvalumab. One of these patients progressed due to an increase in the target lesion, but had a subsequent CR per BICR, which was maintained through the last follow-up as of DCO on Day 650.

### Progression-Free Survival (PFS)

The median PFS in the Primary Efficacy Population was 2.2 months (95% CT: 1.4, 2.7). The PFS rate at 6 months was 28.3% (95% CI: 19.3%, 37.9%).

### Overall Survival (OS)

The OS results were encouraging in the heavily pretreated UC population. Median OS was 14.1 months (95% CI: 4.5, not estimable); the OS rate at 6 months, 9 months, and 12 months was 60.3% (95% CI: 48.7%, 70.1%), 55.7% (95% CI: 43.2%, 66.4%), and 52.4% (95% CI: 39.1%, 64.1%), respectively.

### Clinical Activity in Subgroups

Clinical activity in the Primary Efficacy Population was seen across PD-L1 high and PD-L1low/negative subgroups (as defined by TC or IC 25% PD-L1 expression using the validated VENTANA PD-L1 [SP263] assay) and across various baseline disease subgroups, including those with poor prognosis. In the PD-L1 high subgroup, the ORR was 29% (18/61; 95% CI: 18.5%, 42.6%). In the PD-L1 low/negative subgroup, the ORR was 7.7% (3/39; 95% CT: 1.6%, 20.9%). CRs were observed in both subgroups: PD-L1 high, 3 (4.9%) and PD-L1 low/negative, 2 (5.1%). ORRs were 19.4% (19/98; 95% CI: 12.1%, 28.6%), 10% (5/50; 95% CI: 3.3%, 21.8%) and 40% (2/5; 95% CI: 5.3%, 85.3%) in the visceral metastasis, liver metastasis, and lymph node only subgroups, respectively.

Compelling efficacy and safety data from the UC cohort (UC target population) of Study 1108 support the data disclosed herein for use of durvalumab in the treatment of patients with locally advanced or metastatic bladder cancer, e.g., UC, whose disease has progressed during or after a primary standard platinum-based regimen. In the UC cohort, a therapeutic regimen of durvalumab dosed at 10 mg/kg Q2W resulted in an ORR of 20.4% (21/103; 95% CI: 13.1%, 29.5%), a meaningful improvement over commonly used therapies. The responses occurred early during treatment and were durable. Among the 21 responders, the median time to response was 1.41 months (range, 1.2 to 7.2). The median DoR was not reached (range, 1.4 to 19.9+ months); however, 16 patients remained in response for at least 6 months, and 7 patients remained in response for at least 9 months. Eighteen of the 21 responders (85.7%) had ongoing responses at DCO. The 3 responders who subsequently progressed according to RECIST v1.1 criteria had completed the entire 12-month course of durvalumab therapy and remain alive.

Responses were observed in both PD-L1 high (29.5%; 18/61 [95% CI: 18.5%, 42.6%]) and PD-L1 low/negative subgroups (7.7%; 3/39 [95% CI: 1.6%, 20.9%]), with durability data comparable to the total UC cohort. Median OS in the UC cohort was 14.1 months, with OS rates at 6 and 9 months of 60.3% and 55.5%, respectively. These rates generally exceeded the survival benefits from post-platinum chemotherapies. Among the UC cohort, common AEs (≥10%) were consistent with the underlying disease and included fatigue, constipation, decreased appetite, nausea, anemia, back pain, pyrexia, urinary tract infection, diarrhea, peripheral edema, dyspnea, and vomiting. The incidence of treatment-related Grade 3 or 4 AEs (5.2%), and treatment-related AEs resulting in death (1.0%) or permanent discontinuation of durvalumab (3.7%) was low and consistent across the patient populations, supporting a well-tolerated safety profile of this regimen for treating bladder cancer such as UC. Two treatment-related deaths occurred, both due to an autoimmune hepatitis and pneumonitis (imAE).

Important risks associated with the use of durvalumab (i.e., immune-mediated pneumonitis, hepatitis, diarrhea or colitis, endocrinopathies (e.g., hyperthyroidism, hypothyroidism, adrenal insufficiency, and hypopituitarism, type 1 diabetes mellitus), nephritis, dermatitis or rash, and infusion-related reactions) occurred at low rates, were generally low severity, and were manageable through established treatment guidelines. In the UC cohort, 89 (46.6%) patients had an adverse event of special interest (AESI), and 15 (7.9%) patients had events identified as imAEs. One patient permanently discontinued durvalumab due to an imAE. The most common imAEs (>1% of patients) were in the categories of hypothyroidism (3.7%) and select hepatic events (1.6%). Three patients had a Grade 3 or 4 imAE, including increased ALT, increased AST, increased transaminases, maculo-papular rash, and acute kidney injury (tubulointerstitial nephritis).

In accordance with the described methods and results in treating bladder cancer, such as UC, the anti-PD-L1 antibody durvalumab had demonstrated clinical activity (including complete response) and durability of response in both PD-L1 high and PD-L1 low/negative subgroups, with no appreciable differences in the safety profile. Thus, knowledge of PD-L1 expression is not essential for the safe and effective use of durvalumab. However, the PD-L1 status of a patient's tumor may assist in determining the benefits and risks of a patient's treatment options, and may assist in setting therapeutic expectations during the physician and patient dialogue.

Currently, there is no effective or standard second line (2L) therapy for patients with locally advanced or metastatic UC whose disease has progressed following platinum-based therapy. The mainstay of current therapy involves chemotherapy with low ORRs (approximately 10%) and OS ranges of approximately 6 to 9 months, along with considerable toxicities such as neutropenia (7% -83%), anemia (11% -27%), thrombocytopenia (6% -23%), pain (39%), and infections (6%). Rates of clinically important AEs in patients treated with durvalumab compared favorably to commonly used therapies, and the nature and frequency of imAEs were generally aligned with rates in patients treated with atezolizumab.

Treatment of subjects with a bladder cancer such as UC with durvalumab at a dose of 10 mg/kg Q2W was found to have a favorable benefit:risk profile in the indication. Both a compelling ORR and a durability of response are two surrogate endpoints determined from the study that are supportive of a clinical benefit and a favorable tolerability profile in an area without available therapy. Given the high unmet medical needs for patients with locally advanced or metastatic UC whose disease has progressed during or after one standard platinum-based regimen, there is a need in the field for the use of durvalumab for treating bladder cancer and UC, which are serious life-threatening diseases with a lack of sound and beneficial treatment options. Thus, the present methods involving durvalumab administered to a subject having bladder cancer at a dose of 10-20 mg/kg Q2W or Q4W, particularly at a dose of 10 mg/kg Q2W, provide a therapeutic regimen that provides clinically beneficial results that are available to bladder cancer patients in need of treatment.

### EXAMPLE 3

### Combination of Durvalumab and Trememlimumab for Treating Bladder Cancer, e.g., UC

A confirmatory clinical trial (DANUBE) supports the clinical study described in Example 2 for the use of durvalumab in the treatment of UC. The supportive clinical study is a Phase 3 randomized, open-label, controlled, multicenter, global trial to determine the efficacy and safety of durvalumab monotherapy (1.5 g IV every 4 weeks (Q4W)); or durvalumab (1.5 g IV Q4W) in combination with tremelimumab (75 mg IV Q4W) for up to 4 doses/cycle each followed by durvalumab (1.5 g IV Q4W) versus Standard-of-Care (SoC): (cisplatin+gemcitabine or carboplatin+gemcitabine doublet, based on cisplatin eligibility) 1L chemotherapy. In this study, patients are treatment-naive with histologically or cytologically documented, unresectable, Stage IV transitional cell carcinoma (transitional cell and mixed transitional/non-transitional cell histologies) of the urothelium (including renal pelvis, ureters, urinary bladder, and urethra). Patients are randomized (1:1:1) to treatment with a durvalumab+tremelimumab combination therapy, with durvalumab monotherapy, or with SoC treatment. The primary objectives are to assess the efficacy of the durvalumab+tremelimumab combination therapy versus SoC treatment in terms of PFS and OS in all UC patients, and to assess the efficacy of durvalumab monotherapy versus SoC treatment in terms of OS in patients with UC who have UC cancer or tumor cells or tissue identified as PD-L1 high (PD-L1-high UC patients). Key secondary objectives are the assessment of the efficacy of durvalumab monotherapy versus SoC treatment in terms of PFS in PD-L1 high UC patients and in terms of PFS and OS in all UC patients. This study has to date randomized 625 patients, and its enrollment is ongoing. PFS with interim OS are analyzed as the study progresses, with a final OS assessment made at the end of the study.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

| Listing of SEQ ID NOs | |
|---|---|
| PD-L1 polypeptide sequence | |
| SEQ ID NO: 1 | |
| MRIFAVFIFM TYWHLLNAPY NKINQRILVV DPVTSEHELT CQAEGYPKAE VIWTSSDHQV | |
| LSGKTTTTNS KREEKLFNVT STLRINTTTN EIFYCTFRRL DPEENHTAEL VIPELPLAHP | |
| PNERTHLVIL GAILLCLGVA LTFIFRLRKG RMMDVKKCGI QDTNSKKQSD THLEET | |
| PD L1 nucleic acid sequence | |
| SEQ ID NO: 2 | |
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

MEDI4736 VL
SEQ ID NO: 3
EIVLTQSPGTLSLSPGERATLSCRASQRVSSSYLAWYQQKPGQAPRLLIYDASSRATGIPDRFSGSGSGTDFTLTIS
RLEPEDFAVYYCQQYGSLPWTFGQGTKVEIK
MEDI4736 VH
SEQ ID NO: 4
EVQLVESGGGLVQPGGSLRLSCAASGFTFSRYWMSWVRQAPGKGLEWVANIKQDGSEKYYVDSVKGRFTISRDNAKN
SLYLQMNSLRAEDTAVYYCAREGGWFGELAFDYWGQGTLVTVSS
MEDI4736 VH CDR1
SEQ ID NO: 5
GFTFSRYWMS
MEDI4736 VH CDR2
SEQ ID NO: 6
NIKQDGSEKYYVDSVKG
MEDI4736 VH CDR3
SEQ ID NO: 7
EGGWFGELAFDY
MEDI4736 VL CDR1
SEQ ID NO: 8
RASQRVSSSYLA
MEDI4736 VL CDR2
SEQ ID NO: 9
DASSRAT
MEDI4736 VL CDR3
SEQ ID NO: 10
QQYGSLPWT
Tremelimumab
SEQ ID NO: 11
PSSLSASVGDRVTITCRASQSINSYLDWYQQKPGKAPKLLIYAASSLQSGVPSRFSGSGSGTDFTLTISSLQPEDFA
TYYCQQYYSTPFTFGPGTKVEIKRTVAAPSVFIFPPSDEQLKSGTASVVCLLNNFYPREAKV
Tremelimumab VH
SEQ ID NO: 12
GVVQPGRSLRLSCAASGFTFSSYGMHWVRQAPGKGLEWVAVIWYDGSNKYYADSVKGRFTISRDNSKNTLYLQMNSL
RAEDTAVYYCARDPRGATLYYYYYGMDVWGQGTTVTVSSASTKGPSVFPLAPCSRSTSESTAALGCLVKDYFPEPVT
VSWNSGALTSGVH
Tremelimumab VH CDR1
SEQ ID NO: 13
GFTFSSYGMH
Tremelimumab VH CDR2
SEQ ID NO: 14
VIWYDGSNKYYADSV
Tremelimumab VH CDR3
SEQ ID NO: 15
DPRGATLYYYYYGMDV
Tremelimumab VL CDR1
SEQ ID NO: 16
RASQSINSYLD
Tremelimumab VL CDR2
SEQ ID NO: 17
AASSLQS
Tremelimumab VL CDR3
SEQ ID NO: 18
QQYYSTPFT

1. A method of treating bladder cancer in a subject in need thereof, the method comprising administering to the subject having bladder cancer durvalumab or an antigen binding fragment thereof in an amount of 50 to 2000 mg every 2-4 weeks (Q2W to Q4W), in combination with administering to the subject tremelimumab or an antigen binding fragment thereof in an amount of 50 to 150 mg every 2 to 4 weeks to treat the bladder cancer.

2. The method of claim 1, wherein the durvalumab or an antigen binding fragment thereof is administered in an amount of 1500 mg every 4 weeks (Q4W) and the tremelimumab or an antigen binding fragment thereof is administered in an amount of 75 mg every 4 weeks (Q4W).

3. The method of claim 1, wherein the durvalumab and the tremelimumab or antigen binding fragments thereof are administered for up to four doses per cycle.

4. The method of claim 1, wherein the durvalumab and the tremelimumab or antigen binding fragments thereof are administered at the same time or at different times.

5. The method of claim 1, wherein the subject is identified as having a bladder cancer with a reduced or low level of expression of PD-L1 (PD-L1-low), or with negligible to low expression of PD-L1 (PD-L1-low/neg).

6. The method of claim 1, wherein the subject is identified as having a bladder cancer with a high level of expression of PD-L1 (PD-L1-high).

7. The method of claim 1, wherein the bladder cancer is urolethial carcinoma (UC) and/or cancer of bladder-associated structures and tissue comprising ureter, urethra, urachus and/or renal pelvis.

8. The method of claim 7, wherein the bladder cancer is urothelial carcinoma, advanced UC, or metastatic UC.

9. The method of claim 1, wherein about 60% of treated subjects have an overall survival response at 12 months.

10. The method of claim 1, wherein about 40% of treated subjects have an overall survival response at 24 months.

11. A method of treating bladder cancer in a subject in need thereof, the method comprising administering to the subject having bladder cancer durvalumab or an antigen binding fragment thereof in an amount of 10 mg/kg to 20 mg/kg every 2-4 weeks (Q2W to Q4W), in combination with administering to the subject tremelimumab or an antigen binding fragment thereof in an amount of 1 mg/kg to 10 mg/kg every 2 to 4 weeks to treat the bladder cancer.

12. The method of claim 11, wherein the tremelimumab or an antigen binding fragment thereof is co-administered to the subject with bladder cancer at the same time or at a different time as the administration of the durvalumab or an antigen binding fragment thereof

13. The method of claim 11, wherein the durvalumab or an antigen binding fragment thereof is administered to the subject in an amount of 10 mg/kg every 2-4 weeks (Q2W to Q4W).

14. The method of claim 11, wherein the durvalumab or an antigen binding fragment thereof is administered to the subject in an amount of 20 mg/kg every 2-4 weeks (Q2W to Q4W).

15. The method of claim 11, wherein the tremelimumab or an antigen binding fragment thereof is administered to the subject in an amount of 1 mg/kg every 2-4 weeks (Q2W to Q4W).

16. The method of claim 11, wherein the tremelimumab or an antigen binding fragment thereof is administered to the subject in an amount of 3 mg/kg every 2-4 weeks (Q2W to Q4W).

17. The method of claim 11, wherein the subject has previously undergone a first line cancer therapy comprising a platinum drug or a platinum drug in combination with another anti-cancer agent.

18. The method of claim 17, wherein the first line cancer therapy comprises a therapy selected from cisplatin, cisplatin and gemcitabine, cisplatin and methotrexate, vinblastine, ADRIAMYCIN (doxorubicin), or carboplatin and gemcitabine doublet combination.

19. A method of treating a subject with bladder cancer whose cancer has progressed after treatment with a first line cancer therapy comprising a platinum drug or a platinum drug in combination with another anti-cancer agent, the method comprising:
administering to the subject who has previously been treated with the first line cancer therapy durvalumab or an antigen binding fragment thereof in an amount of 20 mg/kg every 4 weeks (Q4W), wherein the anti-PD-L1 antibody or an antigen binding fragment thereof is durvalumab or an antigen binding fragment thereof,
wherein tremelimumab or an antigen binding fragment thereof in an amount of 1 mg/kg every 4 weeks (Q4W) is co-administered to the subject with bladder cancer at the same time or at a different time as the administration of the anti-PD-L1 antibody or an antigen binding fragment thereof.

20. The method of claim 19, wherein the treatment results in an increase in overall survival, progression free survival, or a complete response in the subject.
